SEQUENCE LISTING (1 sequence)

SEQ ID NO: 1 - DNA, 20 bp, Artificial
  Synthetic
gccttgctgcttcacatgga

(19) United States
(12) Patent Application Publication
(10) Pub. No.: US 2022/0331371 A1
(43) Pub. Date: Oct. 20, 2022

(54) DEVELOPMENT OF EMBRYONIC-LIKE TISSUE FROM STEM CELLS

(71) Applicant: The Regents of the University of Michigan, Ann Arbor, MI (US)

(72) Inventors: Jianping Fu, Ann Arbor, MI (US); Yi Zheng, Ann Arbor, MI (US)

(21) Appl. No.: 17/640,585

(22) PCT Filed: Sep. 8, 2020
(86) PCT No.: PCT/US2020/049721

§ 371 Date: Mar. 4, 2022

Related U.S. Application Data
(60) Provisional application No. 62/897,565, filed on Sep. 9, 2019.

(51) Int. Cl. A61K 35/545; A61K 35/50; C12N 5/0735; C12N 5/074

(52) U.S. Cl. CPC A61K 35/545; A61K 35/50; C12N 5/0606; C12N 5/0696; C12N 2501/115; C12N 2501/155

(57) ABSTRACT

The present disclosure provides compositions and methods employing stem cell-derived embryo-like structures. In some embodiments, methods of generating embryo-like tissues from stem cells and the resulting tissues are provided. In some embodiments, uses of such tissues for research, compound screening and analysis, and therapeutics are provided. Accordingly, in some embodiments, provided herein is a method for preparing embryo-like tissue, comprising: a) introducing stem cells into a microfluidic device comprising a culture channel and a plurality of fluidic channels, wherein the stem cells are introduced to the culture channel of the microfluidic device; b) contacting the stem cells with basal medium via the plurality of fluidic channels for at least 18 hours (e.g., 36 hours) to generate the embryo-like tissue.

### CROSS-REFERENCE TO RELATED APPLICATIONS

The present invention claims priority to U.S. Provisional Patent Application 62/897,565, filed Sep. 9, 2019, which is incorporated by reference in its entirety.

### FIELD

The present disclosure provides compositions and methods employing stem cell-derived embryo-like structures. In some embodiments, methods of generating embryo-like tissues from human stem cells and the resulting tissues are provided. In sonic embodiments, uses of such tissues for research, compound screening and analysis, and therapeutics are provided.

### BACKGROUND

Stem cells are cells with remarkable potential to develop into many different cell types during early life and growth. In addition, in many tissues they serve as a sort of internal repair system, dividing essentially without limit to replenish other cells as long as the person or animal is still alive. Stem cells can be either totipotent, pluripotent, or multipotent. Totipotent cells can form all the cell types in a body, plus all the extraembryonic cells. Pluripotent cells can give rise to all of the cell types that make up the body; embryonic stem cells are considered pluripotent. Multipotent cells can develop into more than one cell type, but are more limited than pluripotent cells; adult stem cells are considered multipotent. Induced pluripotent stem cells (iPSCs) are adult cells that have been genetically reprogrammed to an embryonic-like pluripotent state. Totipotent stem cells can also be obtained through reprogramming of embryonic stem cells or iPSCs.

Stem cells carry promises for regenerative medicine and cell therapy, but are also changing the drug discovery and development process. Emergence of stem cell technologies provides new opportunities to build innovative cellular models. Stem cell models offer new opportunities to improve the manner in which pharmaceutical researchers identify lead candidates and bring new drugs to the market. Stem cell models also offer new opportunities to improve drug and toxicity screens to prevent pregnancy failure and birth defects. In spite of promising applications, new competencies surrounding stem cell differentiation and proliferation, induction of totipotent and pluripotent stem cells and creation of efficacy assays are needed to make successful use of stem cells in regenerative medicine, cell therapy and drug development.

Beyond improved models, stem cells technologies are introducing applications that were previously not possible. Currently, human clinical populations are poorly represented in drug development with a lack of genetic heterogeneity in human cellular models and a limited number of human disease models. As a result of iPSC technology, new cellular models can be created from individuals with a diverse range of drug susceptibilities and resistances, offering the promise of a “clinical trial in a dish” in a field where a personalized medicine approach is becoming increasingly predominant.

Despite these advantages there are still several challenges in using stem cells in research and drug development, The differentiation and reprogramming strategies are not standardized and are often based on growth factors, making protocols expensive, poorly reproducible and limited in terms of scale-up. The pace of stem cell research for example, a single differentiation or reprogramming experiment currently can take more than a month—is too slow to fit into timelines required by the industry. In addition, before pharmaceutical companies typically will invest in the development of such platforms, further demonstrations of success and potential applications are often necessary. And last but not least, stem cell culture and differentiation need to be adapted to the high-throughput environment of drug and toxicity screens by developing standardized high-throughput and miniaturized assays for in vitro screening.

### SUMMARY

The present disclosure provides compositions and methods employing stem cell-derived embryo-like structures. In some embodiments, methods of generating embryo-like tissues from human stem cells and the resulting tissues are provided. In some embodiments, uses of such tissues for research, compound screening and analysis, and therapeutics are provided.

Early human embryonic development involves extensive lineage diversification, cell-fate specification and tissue patterning 1. Despite its basic and clinical importance, early human embryonic development remains relatively unexplained owing to interspecies divergence 2,3 and limited accessibility to human embryo samples. Experiments described herein demonstrated that human stem cells, such as human pluripotent stem cells (hPSCs), in a microfluidic device recapitulate, in a highly controllable and scalable fashion, landmarks of the development of the epiblast and amniotic ectoderm parts of the conceptus, including lumenogenesis of the epiblast and the resultant pro-amniotic cavity, formation of a bipolar embryonic sac, and specification of primordial germ cells and primitive streak cells. Further experiments demonstrated that that amniotic ectoderm-like cells function as a signaling center to trigger the onset of gastrulation-like events. Given its controllability and scalability, the microfluidic model and resulting embryo-like tissues provide a powerful experimental system to advance knowledge of human embryology and reproduction, assist in the rational design of differentiation protocols of human stem cells for disease modelling and cell therapy, and in high-throughput drug and toxicity screens to prevent pregnancy failure and birth defects.

Accordingly, in some embodiments, provided herein is a method for preparing embryo-like tissue, comprising: a) introducing stem cells into a microfluidic device comprising a culture channel and a plurality of fluidic channels, wherein the stem cells are introduced to the culture channel of the microfluidic device; b) contacting the stem cells with basal medium via the plurality of fluidic channels for at least 18 hours (e.g., 36 hours) to generate the embryo-like tissue. In some embodiments, prior to additional of basal medium (e.g., time 0), cells are seeded in the device.

The present disclosure is not limited to particular medium conditions. In some exemplary embodiments, the basal medium comprises E6 medium and basic fibroblast growth factor (FGF2). In some embodiments, the basal medium is supplemented with one or more additional components that alter BMP, WNT, YAP, and/or TGF-β signaling (e.g., selected from, for example, Bone Morphogenic Protein 4 (BMP4), noggin, and a Wnt inhibitor (e.g., IWP2)). In some embodiments, the additional components are added to the basal medium after 12 hours (e.g., after 12, 24, 36, 48, or 120 hours). In some embodiments, the contacting is for at least 12 hours (e.g., at least 12, 24, 36, 48, or 120 hours). In some embodiments, the plurality of fluidic channels comprises an induction channel and a cell loading channel. In some embodiments, both the induction channel and the cell loading channel comprise basal medium. In some embodiments, the induction channel comprises basal medium plus BMP4 and the cell loading channel comprises basal medium. In some embodiments, the induction channel comprises basal medium plus BMP4 and the cell loading channel comprises basal medium plus noggin and IWP2. In sonic embodiments, the induction channel comprises basal medium and cell loading channel comprises basal medium plus BMP4. In some embodiments, the induction channel comprises basal medium plus activin and the cell loading channel comprises basal medium plus BMP4. In some embodiments, the basal medium comprises Essential 6 medium and FGF2, Essential 8 medium and FGF2, or mTesR1 medium and FGF2, or N2B27 medium and FGF2.

In some embodiments, the culture channels comprise a plurality of posts and a gel matrix, and wherein the stem cells are located in pockets (e.g., concave pockets) between the posts and the gel matrix.

The present disclosure provides a variety of different embryo-like tissues. For example, in some embodiments, the embryo-like tissue is a posteriorized embryonic-like sac (P-ELS) or an anteriorized embryonic-like sac (A-ELS). In sonic embodiments, the P-ELS comprises a single layer of amniotic ectoderm-like cells at a pole of the sac exposed to BMP4 and a stratified, epiblast-like epithelium comprising pre-primitive steak (Pre-PS)-epiblast cells at a pole exposed to basal medium. In some embodiments, the A-ELS comprises a single layer of amniotic ectoderm-like cells at a pole of the sac exposed to BMP4 and a single layer of embryonic stem cells in an epiblast-like pole exposed to noggin and IWP2. In some embodiments, the amniotic ectoderm-like cells express Transcription Factor AP2 gamma (TFAP2A); the PrePS-epiblast cells express Caudal type Homobox Transcription Factor 2 (CDX2) and T-Box Transcription Factor (T); and the embryonic stem cells in the epiblast-like layer express Octamer-Binding Transcription Factor 4 (OCT4) and Homobox Transcription Factor NANOG (NANOG). In some embodiments, the embryo-like tissue comprises primordial germ cell-like cells. In some embodiments, the primordial germ cell-like cells express one or more of TFAP2C, SRI Box 17 (SOX17), PR Domain Zinc Finger Protein 1 (BLIMP 1), or NANOG. In some embodiments, the embryo-like tissues comprise primitive streak cells. In some embodiments, the embryo-like tissues comprise mesoderm cells or endoderm cells. In some embodiments, the embryo-like tissue comprises one or more of amniotic ectoderm like cells, primitive steak cells, mesoderm cells, endoderm cells, or primordial germ cell-like cells.

The present disclosure is not limited to particular stem cells for use in generating embryo-like tissues. In some embodiments, the embryonic stem cells are induced pluripotent stem cells (iPSCs) (e.g., human iPSCs), pluripotent stem cells, totipotent, stem cells, embryonic stem cells, expanded potential stem cells, trophoblast stem cells, or hypoblast stem cells. In some embodiments, the stem cells are human, non-human primate monkey, or other mammalian (e.g., pig or cow) stem cells.

Additional embodiments provide a method of generating amniotic ectoderm-like cells, comprising: a) introducing stem cells onto a permeable support comprising a porous membrane; b) contacting the stem cells with basal medium plus BMP4 to generate amniotic ectoderm like cells.

Yet other embodiments provide a method of generating primitive steak cells and/or primordial germ cell-like cells, comprising: co-culturing amniotic ectoderm-like cell and pluripotent stem cells under conditions such that the primitive steak cells and/or primordial germ cell-like cells are generated.

Additional embodiments provide a plurality of embryo-like tissues produced by a method described herein.

Further embodiments provide a method for testing a compound, comprising: a) providing an embryo-like tissue described herein; h) exposing a test compound to the composition; and c) determining an effect of the test compound on the composition. The present disclosure is not limited to particular test compounds. In some embodiments, the compound is a candidate fertility drug. In some embodiments, the compound is screened for toxicity to an embryo (e.g., the effect is the presence or absence of toxicity).

Certain embodiments provide a composition, kit, or system comprising embryo-like tissue described herein.

Additional embodiments are described herein.

### DESCRIPTION OF THE FIGURES

The patent or application file contains at least one drawing executed in color. Copies of this patent or patent application publication with color drawings will be provided by the Office upon request and payment of the necessary fee.

FIG. 1 shows microfluidic modelling of human epiblast and amnion development. a, Mid-sagittal view of the post-implantation human embryo. b, Microfluidic generation of epiblast-like cysts, A-ELS and P-ELS. c, Representative confocal micrographs showing epiblast (EPI)-like cysts at t=36 h stained for OCT4, NANOG and SOX2. d, Representative confocal micrographs showing P-ELS at t=36 h stained for TFAP2A, OCT4 and T (top); CDX2, NANOG and T (middle); TFAP2C, NANOG and SOX17 (bottom). e, Representative confocal micrographs showing A-ELS at t=36 h stained for TFAP2A, NANOG and T (top); OCT4 and NANOG (bottom). Scale bars, 40 μm. BM, basal medium.

FIG. 2 shows single-cell transcriptomic analysis of posteriorized embryonic-like sac. a, Left, schematic of P-ELS at t=48 h. Right, representative confocal micrographs showing staining for CDX2, EOMES and T (top); TFAP2C, NANOG and. SOX17 (bottom). b, t-SNE plot generated from scRNA-seq data of a total of 9,966 cells, revealing six distinct, color-coded cell populations (human ES cell, Transwell-AMLC, AMLC, hPGCLC, MeLC1 and MeLC2; see FIG. 11). Cell numbers of each population are indicated. c, Heat map of correlation coefficients among indicated cell types 21,23,25,26.

FIG. 3 shows that amniotic ectoderm-like cells trigger mesoderm induction in posteriorized embryonic-like sac involving Wnt signalling. a, Schematic of microfluidic setting for mesoderm induction in P-ELS. Scale bars, 40 μm. b, Co-culture assay of AMLCs and human ES cells. Scale bars, 160 μm (main panels) and 10 μm (insets). c, Top, live imaging with TCF/Lef:H2B-GFP human ES cell reporter line to track Win-β-catenin signalling dynamics during embryonic-like sac development with or without Wnt inhibitors IWP2 or IWR1 supplemented into the induction channel as indicated. Bottom, representative confocal micrographs showing embryonic-like sacs obtained at t=36 h when indicated antagonists are supplemented into the induction channel. Scale bars, 40 μm.

FIG. 4 shows microfluidic generation of pluripotent epiblast-like cyst. a, Photograph showing microfluidic devices in a six-well plate. Inset shows a top view of the device. h, Design of microfluidic device incorporating three parallel channels (80 μm in height) partitioned by trapezoid-shaped supporting posts spaced 80 μm apart. c, Protocol for generating epiblast-like cysts. d, Schematic showing cell loading, cell clustering and lumenogenesis. e, Representative bright-field images showing an array of epiblast-like cysts at t=0 h and 36 h. f, Representative confocal micrographs showing epiblast-like cysts at indicated time points stained for ezrin (top) or E-cadherin (E-cad) and laminin (Lain; bottom). g, Representative confocal micrographs showing X-Y, X-Z and Y-Z sections of epiblast-like cysts obtained at t=36 h, stained for E-cadherin and ezrin. Scale bars, 40 μm. h, Percentage of epiblast-like cysts with single, multiple or no lumenal cavities at indicated time points. n=59, 57, 60, 89 and 110 cysts for t=0 h, 6 h, 12 h, 24 h and 36 h, respectively. i, Cell number in each epiblast-like cyst as a function of time. Red lines represent the median. j, Equivalent epiblast-like cyst diameter as a function of time. Red lines represent the median. k, Embedded epiblast-like cyst perimeter percentage as a function of time. Red lines represent the median. l, Representative confocal micrographs showing epiblast-like cysts at t=36 h stained for ZO1 and actin (top) or GM130 and actin (bottom). Far right, magnified views of outlined regions. Scale bars, 40 m, Representative confocal micrographs showing epiblast-like cysts obtained at t=36 h with indicated drugs supplemented into basal medium from t=0-36 h. n, Equivalent epiblast-like cyst diameter at t=36 h under indicated. conditions. Red lines represent the median.

FIG. 5 shows progressive development of posteriorized embryonic-like sac. a, Protocol for generating P-ELS. Scale bar, 80 μM. b, Percentage of P-ELS and uniformly squamous amniotic ectoderm-like cysts at t=36 h. Scale bar, 40 μm. Red lines represent the median. c, Cell number in each cyst as a function of time. n=26 cysts for each time point. Red lines represent the median. d, Representative confocal micrographs showing X-Y, X-Z and Y-Z sections of P-ELS at t=36 h stained for E-cadherin and ezrin. Scale bars, 40 μm. e, Representative confocal micrographs showing P-ELS at t=36 h stained for pSMAD1/5 and TFAP2A. Scale bars, 40 μm. f, Representative confocal micrographs showing P-ELS at indicated time points co-stained for NANOG and CDX2 (left) or T and E-cadherin (right). Scale bars, 40 μm. g, Representative confocal micrographs showing amniotic ectoderm-like tissues at t=72 h stained for E-cadherin, GATA3 and OCT4, TFAP2A and T, or TFAP2C and NANOG as indicated. Bottom images show magnified views of amniotic ectoderm-like tissues. Scale bars, 40 μm (small panels) and 160 μm (large panels). h, Thickness of amniotic ectoderm-like tissue as a function of time. Red lines represent the median. i, In situ hybridization images of P-ELS at t=24 h and t=36 h for BMP4 (left) and AXIN2 (right). Scale bars, 40 μm. j, In situ hybridization images of P-ELS at t=36 h for ACTB (top) and B. subtilis dapB (bottom). Scale bars, 40 μm. k, Left, bright-field and fluorescent micrographs showing P-ELS at t=36 h blocking diffusion of fluorescein-labelled dextran (70 kDa) into the cell loading channel. Scale bar. 40 μm. Right, plot showing relative fluorescence intensity within the cell-loading channel and induction channel. Red lines represent the median.

FIG. 6 shows progressive development of anteriorized embryonic-like sac. a, Protocol for generating A-ELS. b, Percentage of A-ELS and uniformly squamous amniotic ectoderm-like cysts at t=36 h. Scale bar, 40 μm. Red lines represent the median. c, Cell number in each cyst as a function of time. n=26 cysts for each time point. Red lines represent the median. d, Representative confocal micrographs showing A-ELS at t=36 h stained for pSMAD1/5 or TFAP2A. Scale bars, 40 μm. e, Representative confocal micrographs showing A-ELS at indicated time points stained for NANOG, E-cadherin and I. Scale bars, 40 μm. f, Thickness of amniotic ectoderm-like tissue in A-ELS as a function of time. Red lines represent the median.

FIG. 7 shows specification of human primordial germ cell-like cells in posteriorized embryonic-like sac. a, Specification of PGCs or PGCLCs in the M. fascicularis embryo (left; 21) and P-ELS (right). b, Representative confocal micrographs showing P-ELS stained for TFAP2C, NANOG and SOX17 or BLIMP1 and SOX17 at indicated time points. TFAP2C⁺SOX17⁻ and SOX17⁺TFAP2C⁻ cells are marked by green arrows. TFAP2C⁺SOX17⁺ hPGCLCs in the CEN-AM, EPI-AM and epiblast-like compartments are marked by blue, yellow and white arrowheads, respectively. Scale bars, 40 μm. c, Left, dot plot of the numbers of TFAP2C⁺ and SOX17⁺ cells at indicated time points. Right, dot plot of the numbers of TFAP2C⁺SOX17⁺ and TFAP2C⁺ NANOG⁺X17⁺ hPGCLCs at indicated time points. Red lines represent the median. d, Left, spatial distribution of TFAP2C⁺ and SOX17⁺ cells at indicated time points. Right, spatial distribution of TFAP2C⁺SOX17⁺ and TFAP2C^{'0} NANOG⁺SOX17⁺ hPGCLCs at indicated time points. e, Representative confocal micrographs showing P-ELS at t=36 h stained for TFAP2C, T and SOX17. T-expressing (TFAP2C⁺SOX17⁺T⁺) and T-non-expressing (TFAP2C⁺SOX17³⁰ T⁻) hPGCLCs are marked by blue and green arrowheads, respectively. Scale bars, 40 μm. f, Representative confocal micrographs showing P-ELS treated with different doses of the GP130 inhibitor SC144 (top, 0.4 μM; bottom, 2 μM). TFAP2C⁺SOX17⁺ hPGCLCs in the EPI-AM and epiblast-like compartments are marked by yellow and white arrowheads, respectively. Scale bars, 40 μm.

FIG. 8 shows microfluidic modelling of human epiblast and amnion development using HI human ES cells and human induced pluripotent stem cells (hiPSCs) maintained in mTeSR medium as well as H9 human ES cells maintained in Essential 8 medium (E8-H9). a, Microfluidic generation of epiblast-like cysts. b., Microfluidic generation of P-ELS. Representative confocal micrographs show P-ELS generated from H1 human ES cell, hiPSC and E8-H9 human ES cell at t=36 h stained for TFAP2A, OCT4 and T (top); CDX2, NANOG and T (middle); TFAP2C, NANOG and SOX17 (bottom).

TFAP2C⁺NANOG⁺SOX17⁺ hPGCLCs are marked by white arrowheads. c, Microfluidic generation of A-ELS. Representative confocal micrographs show A-ELS generated from H1 human ES cell, hiPSC and E8-H9 human ES cell at t=36 h stained for OCT4 and NANOG (top); TFAP2A, NANOG and T (bottom). Scale bars, 40 μm.

FIG. 9 shows that exogenous Wnt or activin alone are insufficient to generate asymmetric embryonic-like sacs. a, Representative confocal micrographs showing cysts stained for CDX2, EOMES and or OCT4 and NANOG. b, Representative confocal micrographs showing cysts stained for CDX2, EOMES and T or OCT4 and NANOG. c, Representative confocal micrographs showing cysts stained for CDX2, NANOG and T or TFAP2A and I. d, Representative confocal micrographs showing cysts stained for CDX2, EOMES and T or TFAP2A and T. Scale bars, 40 μm.

FIG. 10 shows molecular characterization of posterior and anterior primitive streak-like cell development. a, Schematic showing posterior primitive streak-like cell development in P-ELS at t=48 h with BMP4 (50 ng ml⁻¹) supplemented into basal medium in the cell loading channel. Right, bright-field image shows an array of P-ELS at t=48 h. Scale bar, 80 μm. 1), Representative confocal micrographs show P-ELS stained for E-cadherin and N-cadherin at indicated time points. c, Dot plot of the thickness of the epiblast-like tissue at indicated time points. Red lines represent the median. d, Representative confocal micrographs showing P-ELS stained for CDX2, EOMES and T at indicated time points. e, Representative confocal micrographs show P-ELS stained for TFAP2C, NANOG and SOX17 (left) or BLIMP1 and SOX17 (right) at t=48 h. f, Schematic showing anterior primitive streak-like cell development in P-ELS at t=48 h with BMP4 (50 ng ml⁻¹) and activin A. (50 ng ml⁻¹) supplemented into the cell-loading and induction channels, respectively. Right, bright-field image shows an array of P-ELS at t=48 h. Scale bar, 80 μm. g, Representative confocal micrographs showing staining for E-cadherin, N-cadherin, CDX2, EOMES and T at indicated time points. h, Representative confocal micrographs show staining, for CDX2, EOMES and T at t=48 h. i, Representative confocal micrographs show staining for TFAP2C, NANOG and SOX17 at t=48 h. Scale bars, 40 μm.

FIG. 11 shows cell-type identification and characterization using scRNA-seq, a, Workflow. b, t-SNE plot generated from scRNA-seq data of a total of 9,966 cells, revealing six distinct, color-coded cell populations (human ES cell. Transwell-AMLC, AMLC, hPGCLC, MeLC1 and MeLC2). c, Violin plots of log-transformed, normalized expression levels of genes associated with pluripotency (POU5F1 (also known as OCT4), SOX2, NANOG, PODXL and DPPA4), hPGC (SOX17, TFAP2C, NANOS3, BLIMP1 and PDPN), amniotic ectoderm (TFAP2A, GATA3, HAND1, TCIM (also known as C8orf4) and IGFBP3), mesoderm (T, EOMES, MIXL1, LHX1,MESP2, MESP1, GATA6, LEF1, CDX2 and SNA12) and HOX proteins (HOXB6, HOXB7, HOXB8, HOXB9 and HOXA10) in the six cell populations as indicated. d, Heat map of relative expression (Z-score) of top-20 gene signatures distinguishing each cell population. e, DEGs between different cell clusters (MeLC1 against human ES cell; MeLC2 against MeLC1 hPGCLC against human ES cell; AMLC against human ES cell; AMLC against Transwell-AMLC). Top, red and blue bars indicate the numbers of up- and downregulated DEGs, respectively, in indicated pairwise comparisons. Bottom, enrichment of GO terms and representative genes in DEGs from indicated pairwise comparisons. f. Heat map of log-transformed expression levels of selected genes among indicated cell types 21,23. Left, comparisons among M. fascicularis epiblast (CyEPI) lineage, human ES cell and MeLC1. Right, comparisons among human ES cell, AMLC and Transwell-AMLC. g, Heat map of correlation coefficients among indicated cell types 21,23,25,26.

FIG. 12 shows the inductive effect of amniotic ectoderm-like cells on the onset of gastrulation-like events. a, Schematic showing P-ELS; the PrePS-EPI-like compartment is divided into four quadrants (R1, R2, R3 and R4) for quantification. b, Fluorescent and composite images showing dynamic T expression in the PrePS-EPI-like compartment at indicated time points. c, Top, dot plots of relative T intensity in different quadrants of the PrePS-EPI-like compartment at indicated time points. Red lines represent the median. Bottom, spatial maps of average relative T intensity at indicated time points. d, Live imaging with T-mNeonGreen human ES cell reporter line to track dynamic T expression in the PrePS-EPI-like compartment during the development of P-ELS. e, Characterization of T-mNeonGreen human ES cell reporter line showing co-localization of neon green signal and immunostaining of T. f, Representative confocal micrographs showing development of AMLCs by culturing human ES cells on Transwell membranes in basal medium supplemented with BMP4 (50 ng ml⁻¹) for 48 h. Cells were stained for CDX2, NANOG and T (top) or TFAP2A and OCT4 (bottom). g, Representative confocal micrographs showing AMLCs stained for CDX2, NANOG and T (top) or TFAP2A and OCT4 (bottom). h, Transwell co-culture assays of AMLCs and human ES cells. Representative confocal micrographs show staining for CDX2, NANOG and T (top); TFAP2A, OCT4 and (middle); E-cadherin, N-cadherin and T (bottom). i, Transwell co-culture assays of AMLCs and human ES cells. Representative confocal micrographs show staining of cells on the lower dish for OCT4 and T (top); NANOG and T (middle); TFAP2C, NANOG and SOX17 (bottom), or cells on the Transwell membrane for TFAP2C, NANOG and SOX17 as indicated. Boxed images show magnified views of selected areas. In h, insets show human ES cell colonies seeded after AMLC differentiation as marked by white arrowheads. Scale bars in b, d, e: 40 μm. Scale bars in 160 μm (main panels) and 10 μm (insets).

FIG. 13 shows induction of MeLC in posteriorized embryonic-like sac is inhibited by IWP2 but not by IWR1. a, Schematic shows Transwell co-culture protocol. Representative confocal micrographs showing staining for CDX2. NANOG and T (top) or TFAP2A and OCT4 (bottom). Insets show human ES cell colonies seeded after AMLC differentiation, as marked by white arrowheads. Scale bars: 160 μm (main panels) and 10 μm (insets). b, Live imaging with the TCF/Lef:H2BGFP H9 human ES cell reporter line to track Wnt-β-catenin signaling dynamics during embryonic-like sac development with or without the Wnt inhibitors IWR1 (10 μM) or IWP2 (5 μM) supplemented into the induction channel as indicated. Scale bars, 40 μm. c, Representative confocal micrographs show P-ELS obtained at t=48 h with IWR1 (10 μM) supplemented into the induction channel as indicated. P-ELS were stained for CDX2, EOMES and T (top) or TFAP2C, NANOG and SOX17 (bottom). Outlined regions are magnified in the panel below. Scale bars, 40 μm.

FIG. 14 shows an exemplary device used in embodiments of the present disclosure.

### DEFINITIONS

To facilitate an understanding of the present technology, a number of terms and. phrases are defined below. Additional definitions are set forth throughout the detailed description.

Throughout the specification and claims, the following terms take the meanings explicitly associated herein, unless the context clearly dictates otherwise.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the tem' “embryo” refers to a fertilized egg in the process of development for example a human offspring during the period from approximately the second to the eighth week after fertilization.

As used herein, the term “embryo-like tissue” refers to tissue differentiated in vitro (e.g., from a stem cell) that has one or more properties of an embryo (e.g. one or more of primordial germ cell-like cells, amniotic ectoderm-like cells, and an epiblast-like epithelium). However, embryo-like tissue lacks all properties of an embryo and is generally unable to develop beyond the embryo stage. In some embodiments, embryo-like tissue lacks a primitive endoderm. and/or trophoblast.

The term “administration” and variants thereof (e.g., “administering” a compound) in reference to cells or a compound means providing the cells or compound or a prodrug of the compound to the individual in need of treatment or prophylaxis. When cells or a compound of the technology or a prodrug thereof is provided in combination with one or more other active agents, “administration” and its variants are each understood to include provision of the compound or prodrug and other agents at the same time or at different times. When the agents of a combination are administered at the same time, they can be administered together in a single composition or they can be administered separately. As used herein, the term “composition” is intended to encompass a product comprising the specified ingredients in the specified amounts, as well as any product that results, directly or indirectly, from combining the specified ingredients in the specified amounts.

By “pharmaceutically acceptable” is meant that the ingredients of the pharmaceutical composition are compatible with each other and not deleterious to the recipient thereof.

The term “subject” as used herein refers to an animal, preferably a mammal, most preferably a human, who has been the object of treatment, observation, or experiment.

The term “effective amount” as used herein means that amount of an agent (e.g., amnion-like tissue) that elicits the biological or medicinal response in a cell, tissue, organ, system, animal, or human that is being sought by a researcher, veterinarian, medical doctor, or other clinician. In some embodiments, the effective amount is a “therapeutically effective amount” for the alleviation of the symptoms of the disease or condition being treated. In some embodiments, the effective amount is a “prophylactically effective amount” for prophylaxis of the symptoms of the disease or condition being prevented.

### DETAILED DESCRIPTION

The present disclosure provides compositions and methods employing stem cell-derived embryo-like structures. In some embodiments, methods of generating embryo-like tissues from human stem cells and the resulting tissues are provided. In some embodiments, uses of such tissues for research, compound screening and analysis, and therapeutics are provided. In vitro cultured human embryos provide insights about the self-organizing properties and autonomy of early human development 4,5. However, protocols for in vitro human embryo culture beyond the blastocyst stage remain suboptimal 4,5. Furthermore, bioethical guidelines prohibit in vitro culture of human embryos beyond 14 days post fertilization or reaching the onset of primitive streak (PS) development 6,7. Human and mouse pluripotent stem cells in a developmental state similar to the epiblast have been used for modelling post-implantation development of human and mouse embryos 8-16. Thus, models based on stem cells are an important alternative to the use of natural conceptus 17. However, beyond S phenomenological observations, some existing models suffer from unsatisfactory efficiency and reproducibility and are therefore suboptimal for mechanistic studies 8,9,11,15. Recent efforts have made use of micropatterned surfaces 10,13 and microwell structures 14,16 to promote multicellular self-organization in controlled environments.

Described herein is the use of microfluidics to achieve a controllable model system to recapitulate developmental events reflecting epiblast and amniotic ectoderm development in the post-implantation human embryo, Embryo-like tissue generated using the methods described herein finds use in a variety of research, screening, and clinical applications.

### I. Generation of Embryo-Like Tissue

As described herein, the present disclosure provides compositions and methods for generating and utilizing embryo-like tissue.

### Cells

A wide variety of cells and stem cells may be employed with the technology described herein. Such cells include pluripotent stem cells (embryonic stem cells and induced pluripotent stem cells) and totipotent stem cells, regardless of source or species. For example, induced pluripotent stem cells may be derived from stem cells or adult somatic cells that have undergone a dedifferentiation process. Pluripotent stem cells and totipotent stem cells may be human cells or be associated with other species (for example, monkey, pig and cow). induced pluripotent stem cells may be generated using any known approach. in some embodiments, iPSCs are obtained from adult human cells (e.g., fibroblasts). in some embodiments, modification of transcription factors (e.g., Oct3/4, Sox family members (Sox2, Sox1, Sox3, Sox15, Sox18), Klf Family members (Klf4, Klf2, Klf1, Klf5), Myc family members (c-myc, n-myc, l-myc), Nanog, LIN28, Glis1, etc.) or mimicking their activities is employed to generate iPSCs (using transgenic vector (adenovirus, lentivirus, plasmids, transposons, etc.), inhibitors, delivery of proteins, microRNAs, etc.).

Totipotent stem cells may be generated using any known approach. In some embodiments, totipotent stem cells are obtained from pluripotent stem cells (for example, embryonic stem cells). In some embodiments, modification of transcription factors (e.g., Oct3/4, Sox family members (Sox2, Sox1, Sox3, Sox15, Sox18), Klf Family members (Klf4, Klf2, Klf1, Klf5), Myc family members (c-myc, n-myc, l-myc), Nanog, LIN28, Glis1, etc.) or mimicking their activities is employed to generate totipotent stem cells (using transgenic vector (adenovirus, lentivirus, plasmids, transposons, etc.), inhibitors, delivery of proteins, microRNAs, etc.

In some embodiments, the cells are expanded potential stem cells (Yang et al., Nature volume 550, pages 393-397(2017); herein incorporated by reference in its entirety), trophoblast stem cells (Roberts et al., Biol Reprod. 2011 Mar; 84(3): 412-21; herein incorporated by reference in its entirety), or hypoblast stem cells (Nigro et al., Journal of Molecular Cell Biology, Volume 4, issue 6, December 2012, Pages 423-426; herein incorporated by reference in its entirety).

In some embodiments the cells are not human cells and are associated with other mammalian species (for example, monkey, pig, or cow).

In some embodiments the cells are non-terminally differentiated cells (regardless of pluripotency) or other non-maturated cells.

In some embodiments, cells are screened for propensity to develop teratomas or other tumors (e.g., by identifying genetic lesions associated with a neoplastic potential). Such cells, if identified and undesired, are discarded.

### Preparing Tissue

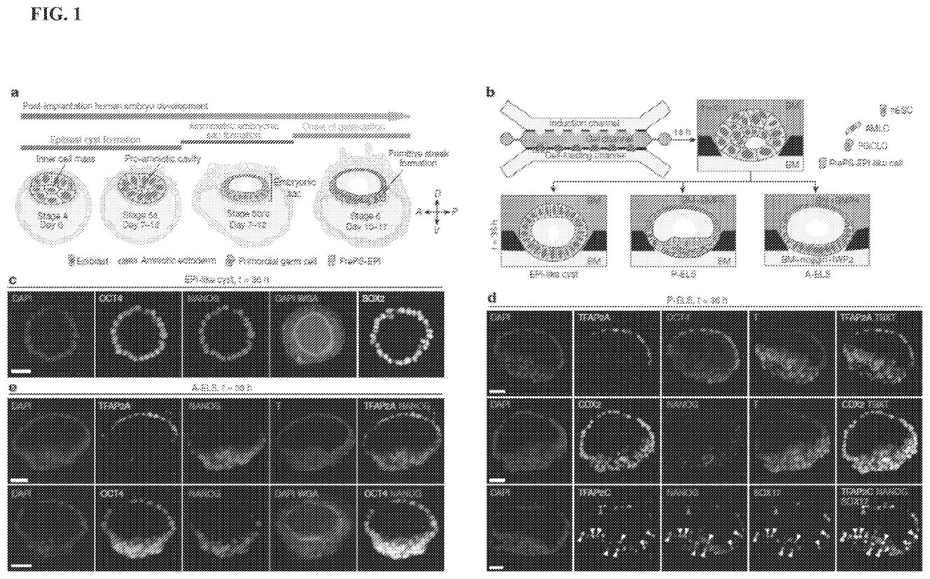

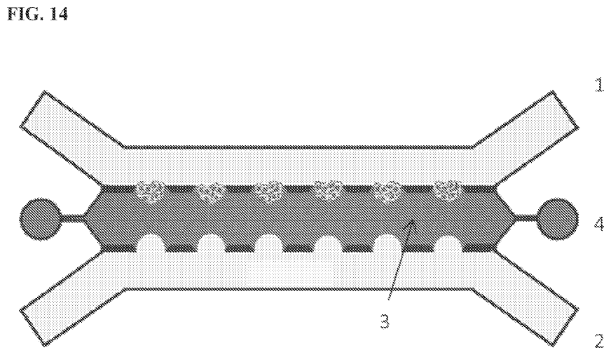

In some embodiments, embryo-like tissues are prepared using a method described herein. For example, in some embodiments, cells are cultured in a microfluidic device comprising a culture channel and a plurality of fluidic channels. In some embodiments, the fluidic channels comprise an induction channel and a cell loading channel. Exemplary devices are shown in FIG. 1b, 14 and described in WO 2018/106997; herein incorporated by reference in its entirety. For example, as shown in FIG. 14, in some embodiments, cells are generated in a device comprising parallel first, second, and third channels wherein the first channel 1 and second channel 2 are cell channels comprising a loading reservoir 4 operably linked to the third channel 3 comprising a gel matrix. Exemplary devices are shown in FIG. 14. As shown in FIG. 14, the cell culture channel 1 (e.g., first channel) is in fluid communication with the cell induction channel 3 (e.g., second channel) via the gel channel (third channel) 3.

In some embodiments, the cell culture channels comprises a plurality of posts and a gel matrix. In sonic embodiments, the gel matrix forms pockets (e.g., concave pockets) between the posts and the gel matrix. In some embodiments, cells are cultured in the pockets.

The present disclosure is not limited to particular gel matrices. In some embodiments, the gel matrix is a natural or synthetic polymeric hydrogel (e.g., polyethylene glycol (PEG) hydrogels, poly (2-hydroxyethyl methacrylate) (PHEMA) hydrogels, growth factor basement membrane matrix, gelatinous protein mixture secreted by Engelbreth-Holm-Swarm (EHS) mouse sarcoma cells (Matrigel hydrogel), collagen, hyaluronic acid (HA), fibrin, or a combination thereof). In some embodiments, commercially available matrices e.g., available from Fisher Scientific (Waltham, Mass.), Amsbio (Abingdon, UK), Coming (Corning, N.Y.), or Trevigen, Inc. (Gaithersburg, Md.) are utilized.

In some embodiments, one or more buffers and/or induction reagents are used to direct differentiation of the cells into a variety of embryo-like tissues. As described herein, a variety of embryo-like tissues are differentiated from human stem cells (e.g., human iPSCs). As described in Example 1, in some embodiments, epiblast-like cysts (ELS) are generated by culturing stem cells in the presence of basal medium. In some embodiments ELSs are directed to develop into either a posteriorized embryonic-like sac (P-ELS) or an anteriorized embryonic-like sac (A-ELS) through the use of additional induction reagents, Both A-ELS and P-ELS are asymmetrical cysts with amniotic ectoderm-like cells (AMLCs) mimicking an amniotic ectoderm at one pole and epiblast-like epithelium mimicking an epiblast at the opposite pole. In the case of P-ELS, the epiblast cells are PrePS-epiblast cells, In the case of A-ELS, the epiblast cells are an organized epiblast-like pole.

The present disclosure is not limited to a particular basal medium. In some embodiments, basal medium for use herein comprises commercially available Essential 6 (E6) medium (e.g., available from Thermo Fisher Scientific, Waltham, Mass.) and FGF2.

In some embodiments, cells are differentiated in the presence of basal medium for a period of time (e.g., at least 1 hour, at least 18 hours, at least 36 hours, at least 48 hours, at least 120 hours, or longer). In some embodiments, additional induction components are added at time 0, 1 hour, 18 hours, 24 hours, 36 hours, 48 hours, 120 hours, or another time point, where time zero is when cells are first contacted with basal medium. In some embodiments, cells are seeded in the device prior to addition of basal medium (e.g., for a period of 1 to 18 hours).

In some embodiments, buffers and induction components are added via one or more or all of the fluidic channels. In some embodiments, different or the same components are added to each of the fluidic channels.

In some embodiments, in order to generate P-ELS, the induction channel comprises basal medium plus BMP4 and the cell loading channel comprises basal medium.

In some embodiments, in order to generate A-ELS, induction channel comprises basal medium plus BMP4 and the cell loading channel comprises basal medium plus noggin and IWP2.

In sonic embodiments, to generate posterior primitive streak cells, the induction channel comprises basal medium and the cell loading channel comprises basal medium plus BMP4. In some embodiments, to generate anterior primitive streak cells, the cell loading channel comprises basal medium plus BMPs in the loading channel, and induction channel comprises activin and basal medium.

In some embodiments, the basal medium comprises Essential 6 medium and FGF2, Essential 8 medium and FGF2, or mTesR1 medium and FGF2, or N2B27 medium and FGF2.

In some embodiments, the medium is changed at a regular interval (e.g., every hour to every day to every week). In some embodiments, medium is changed daily.

In some embodiments, molecular markers are used to verify the presence of a particular embryo-like tissue or cell. For example, in some embodiments, amniotic ectoderm-like cells express TFAP2A; PrePS-epiblast cells express CDX2 and T; and embryonic stem cells in an epiblast-like pole express OCT4 and NANOG.

In some embodiments, the embryo-like tissue comprises primordial germ cell-like cells. Primordial germ cells are the common origins of spermatozoa and oocytes. In some embodiments, the embryo-like tissue described herein comprise primordial germ cell-like cells in the epiblast-like pole and/or the amnion ectoderm-like pole. In some embodiments, the primordial germ cell-like cells express one or more of TFAP2C, SOX17, BLIMP1, and NANOG.

In some embodiments, the embryo-like tissue comprises a primitive streak-like tissue. The primitive streak is an elongated band of cells that forms along the axis of an embryo early in gastrulation by the movement of lateral cells toward the axis and that develops a groove along its midline through which cells move to the interior of the embryo to form the mesoderm. In some embodiments, the primitive streak-like tissue or cells are located in the epiblast like pole or other location.

Additional embodiments provide a method of generating amniotic ectoderm-like cells, comprising: a) introducing stem cells onto a permeable support comprising a porous membrane; b) contacting the stem cells with basal medium plus BMP4 to generate amniotic ectoderm like cells.

Yet other embodiments provide a method of generating primitive steak cells and/or primordial germ cell-like cells, comprising: co-culturing amniotic ectoderm-like cell and pluripotent stem cells under conditions such that the primitive steak cells and/or primordial germ cell-like cells are generated.

### II. Uses

The embryo-like tissues provided herein find use in a variety of research, diagnostic, and therapeutic applications.

In some embodiments, tissues are utilized in research applications (e.g., study of normal or abnormal embryo development). In some embodiments, tissues are used in gene expression analysis to identify genes involved in embryonic development. In some embodiments, tissues are used to identify polymorphisms or mutations involved in defects in development (e.g., defects that may be associated with infertility or miscarriage).

In some embodiments, the tissues are used for disease modeling and drug development. The quality of the embryo-like tissues and the ability to generate them in a short period of time makes them ideally suited for such research uses, particularly high-throughput analysis. Agents are contacted with the cells to determine the effect of the agent. Cells derived from embryo-like tissues may also be modified to include a marker and used either in vitro or in vivo as diagnostic compositions to assess properties of the cells in response to changes in the in vitro or in vivo environment.

In some embodiments, embryo-like tissues or cells derived from embryo-like tissues are used in drug testing or drug toxicity screening applications. For example, in some embodiments, drugs or biological or environmental agents are tested. Indications for drug testing include any compound or biological agent in the pharmaceutical discovery and development stages, or drugs approved by drug regulatory agencies, like the US Federal Drug Agency. All classes of drugs, over-the-counter and nutraceuticals for any medical indications are known or suspected environmental toxicant may be utilized. In some embodiments, drugs are screened using the embryo-like tissues or cells derived from embryo-like tissues described herein to identify drugs that are potentially toxic to embryos or fetuses or general toxicity. In some embodiments, candidate infertility drugs are screened using the embryo-like tissues or cells derived from embryo-like tissues described herein to identify drugs to treat infertility (e.g., by promoting embryonic development, germ-line cell development, etc.).

In some embodiments, screening methods are high throughput screening methods.

In some embodiments, the embryo-like tissues or cells derived from embryo-like tissues find use in therapeutic applications. For example, in sonic embodiments, primordial germ cell-like cells find use in the generation of sperm or egg cells (e.g., from an iPSC from a donor). Such cells find use in research and in therapeutic (e.g., treatment of infertility) uses.

Embodiments of the present disclosure provide kits comprising the cells or tissues described herein. For example, in some embodiments, kits comprise cells or tissues (e.g., embryo-like tissues or human pluripotent stem cells). In some embodiments, kits further comprise reagents for differentiation or use of the cells or tissues described herein (e.g., buffers, test compounds, controls, etc.).

In some embodiments, provided herein are high-throughput systems for generating and/or performing assays with the embryo-like tissues or cells derived from embryo-like tissues described herein. For example, in some embodiments, high-throughput systems comprise devices with a plurality (e.g., 8, 16, 24, 128, etc.) fluidly isolated regions that permit generation and testing of embryo-like tissues in isolated zones, In sonic embodiments, such systems further comprise assay reagents, detection systems, and the like. In some embodiments, systems include automated reagent delivery and/or analysis systems (e.g., robotic sample handling systems).

### EXAMPLES

The following examples are provided in order to demonstrate and further illustrate certain preferred embodiments and aspects of the present disclosure and are not to be construed as limiting the scope thereof.

### Example 1

### Methods

Cell lines. hPSC lines used in this study include H1 human ES cell (WA01, WiCell; NIH registration number: 0043), H9 human ES cell (WA09, WiCell; NIH registration number: 0062) and 1196a (a human iPSC line from the University of Michigan Pluripotent Stem Cell Core31). All hPSC lines have been authenticated by original sources as well as in-house by immunostaining for pluripotency markers and successful differentiation to the three germ layers. All hPSC lines are maintained in a feeder-free system for at least ten passages and authenticated as karyotypically normal. Karyotype analysis was performed by Cell Line Genetics. All hPSC lines are tested negative for mycoplasma contamination (LookOut Mycoplasma PCR Detection Kit, Sigma-Aldrich).
Cell culture. hPSCs were maintained in a standard feeder-free culture system using mTeSR medium (mTeSR; STEMCELL Technologies) or TeSR-E8 medium (Essential 8 or E8; STEMCELL Technologies) and lactate dehydrogenase-elevating virus (LDEV)-free, human ES cell-qualified reduced growth factor basement membrane matrix Geltrex (Thermo Fisher Scientific; derived from Engelbreth-Holm-Swarm tumors similarly to Matrigel). Cell cultures were visually examined during each passage to ensure absence of spontaneously differentiated, mesenchymal-like cells in culture. All hPSCs were used before reaching P70.
Device fabrication. The microfluidic device consists of a polydimethylsiloxane (PDMS) structure layer bonded to a coverslip. The PDMS structure layer is made by mixing PDMS curing agent and base polymer (Sylgard 184; Dow Coming) at a ratio of 1:10 before casting PDMS prepolymer onto a microfabricated silicon mold and baking at 110° C. for 40 min. Medium reservoirs (8 mm in diameter) and gel-loading ports (1.2 mm in diameter) were then punched into the PDMS structure layer using Harris Uni-Core punch tools (Ted Pella). After cleaning with ethanol and air plasma activation, the PDMS structure layer was bonded to a coverslip before baking at 80° C. overnight. Before usage, the microfluidic device was sterilized under UV light for 30 min. Geltrex diluted in mTeSR (8 mg ml⁻¹) was then injected into the central gel channel and allowed to cure for 10 min at 37° C. in an incubator. The central gel channel was separated from the cell loading and induction channels by trapezoid-shaped supporting posts. Diluted Geltrex matrix was contained in the gel channel by supporting posts owing to surface tension. Upon gelation, Geltrex matrix contracts, generating concave Geltrex pockets between supporting posts. mTeSR medium was immediately added to medium reservoirs to fill both the cell loading and induction channels. The microfluidic device was then incubated at 37° C. and 5% CO₂, for 24 h to stabilize the Geltrex matrix in the gel channel.
Microfluidic assays. Colonies of hPSCs were dissociated by Accutasc. (Sigma-Aldrich) at 37° C., for 10 min before being suspended in mTeSR as single cells. Cells were then centrifuged and resuspended in mTeSR, containing 10 μM Y27632 (Tocris), a ROCK inhibitor that prevents dissociation-induced apoptosis of hPSCs32, at a concentration of 8×10⁶ cells per ml. After aspirating mTeSR from medium reservoirs, 10 μl hPSC suspension was introduced into the cell-loading channel (t=−18 h), The microfluidic device was then tilted 90° for 10 min to allow cell settlement into Geltrex pockets and their clustering and adhesion to Geltrex matrix. Medium reservoirs were then refilled with fresh mTeSR medium containing 10 μM Y27632. At t=0 h, all medium reservoirs were switched to a fresh basal medium comprising Essential 6 medium (E6; Thermo Fisher Scientific) and FGF2 (20 ng ml⁻¹; GlobalStem). Unless noted otherwise, all medium reservoirs and both cell loading and induction channels were filled with basal medium from t=0 h onwards and were replenished daily.

To generate epiblast-like cysts, basal medium was used in all medium reservoirs from t=0 h onwards. To examine involvements of different signaling events in the development of epiblast-like cysts, IWP2 (5 μM; Tocris), LDN 193189 (0.5 μM; Selleckchem), SB 431542 (10 μM; Cayman Chemical) or caspase 3 inhibitor Z-DEVD-FMK (10 μM; BioVision) was supplemented into basal medium. To generate P-ELS, BMP4 (50 ng ml⁻¹; R&D Systems) was supplemented into basal medium in the induction channel from t=0 h onwards. To generate A-ELS, in addition to BMP4 (50 ng ml⁻¹) supplemented into basal medium in the induction channel, noggin (50 ng ml⁻¹; R&D Systems) and IWP2 (5 μM in DMSO; Tocris) were supplemented into basal medium in the cell-loading channel from t=0 h onwards.

To examine the effect of exogenous Wnt or activin on generating asymmetric embryonic-like sacs, WNT3A (50 ng ml⁻¹; R&D Systems), activin A (50 ng ml⁻¹; R&D Systems) and/or BMP4 (50 ng ml⁻¹) were supplemented into basal medium in the induction channel. To examine the development of posterior primitive streak-like cells, from t=0 h onwards, BMP4 (50 ng ml⁻¹) was supplemented into basal medium in the cell-loading channel, and basal medium or basal medium supplemented with IWP2 (5 μM), IWR1 (10 M; Selleckchem and STEMCELL Technologies), or noggin (50 ng ml⁻¹) was loaded into the induction channel. To examine anterior primitive streak-like cell development, in addition to BMP4 (50 ng ml⁻¹) supplemented into basal medium in the cell-loading channel, activin A (50 ng ml⁻¹) was added into basal medium in the induction channel.

Immunocytochemistry. hPSCs were fixed in 4% paraformaldehyde (PFA; buffered in 1× PBS) for 12 h, and permeabilized in 0.1% SDS solution (sodium dodecyl sulphate, dissolved in PBS) for another 3 h. Samples were then blocked in 4% donkey serum (Sigma-Aldrich) at 4° C. for 24 h, followed by incubation with primary antibody solutions at 4° C. for another 24 h. Samples were then labelled with donkey-raised secondary antibodies (1:500 dilution) at 4° C. for 24 h, 4,6-diamidino-2-phenylindole (DAPI; Thermo Fisher Scientific) was used for counterstaining cell nuclei. Alexa Fluor dye-conjugated WGA (Thermo Fisher Scientific) and phalloidin (Invitrogen) were used for visualization of cell membrane and actin microfilaments, respectively. Both primary and secondary antibodies were prepared in 4% donkey serum supplemented with 0.1% NaN₃. Seventy microlitre antibody solutions were added to each medium reservoir for immunostaining. The asymmetric embryonic-like sac structure was assessed by immunocytochemistry to confirm molecular asymmetry at t=36 h (for P-ELS: co-staining for TFAP2A and T; for A-ELS: co-staining for TFAP2A and NANOG).
In situ hybridization. In situ hybridization was performed using the ViewRNA ISH Tissue Assay Kit (1-plex; Thermo Fisher Scientific) according to the manufacturer's instructions. In brief, cystic tissues within the microfluidic device were fixed with 4% PFA for 24 h, before being dehydrated by washing with PBT (0.1% Triton X-100 in PBS) and then a graded series of methanol (25%, 50%, 75% and 100% in PBT; twice in each concentration and 10 min for each wash). Before hybridization, cystic tissues were rehydrated using a reverse-graded series of methanol (75%, 50% and 25% in PBT) before being washed twice with PBS. Proteinase K digestion was conducted for 15 min at 40° C., followed by 4% PFA fixation for 15 min at room temperature. Cystic tissues were hybridized with ViewRNA type 1 probe set for 3 h at 40° C. followed by treatment with PreAmplifier for 30 min at 40° C., Amplifier for 20 :min at 40° C., label probe-AP for 20 min at 40° C., AP-enhancer for 8 min at room temperature, and Fast Red for 35 min at 40° C. Gene-specific probes for human AXIN2 (VA1-10388-VT) and BMP-1 (VA1-18826-VT) were tested in this work. Probes against human ACTB (VA1-10351-VT) and Bacillus subtilis dapB (VF1-11712-VT) were used as positive and negative controls, respectively.
Microscopy. All confocal micrographs were acquired using an Olympus DSUIX81 spinning-disc confocal microscope equipped with an EMCCD camera (iXon X3, Andor). For 3D reconstruction, z-stack images were acquired with a slice thickness of 1 μm. Low-magnification bright-field images were acquired using a Labomed TCM 400 inverted microscope equipped with an UCMOS eyepiece camera (Fisher Scientific). For morphogenetic quantification, bright-field or phase-contrast images were acquired using a Zeiss Observer Z1 microscope (Carl Zeiss MicroImaging) equipped with a monochrome CCD camera (AxioCam, Carl Zeiss MicroImaging). Live imaging was conducted using the Zeiss Observer.Z1 microscope enclosed in an environmental incubator (XL S1 incubator, Carl Zeiss MicroImaging) maintaining cell culture at 37° C. and 5% CO₂.
Enumeration of total cell number. For enumeration of cell number during the development of lumenal cystic tissues, a CAG-H2B-EGFP 1-19 human ES cell line was generated. H2B-eGFP (Addgene plasmid #32610) was PCR amplified, and the PCR product was ligated into the ePiggyBac vector with a constitutively active puromycin selection cassette 34. The plasmid was co-transfected with pCAGPBase (ePiggyBac transposase helper plasmid, a gift from A. H. Brivanlou) using GeneJammer (Agilent Technologies) into H9 human ES cells that were plated at 50,000 cells per cm² 24 h before transfection. Puromycin selection (2 μg ml⁻¹) started at day 2 after transfection for 4 days. The brightest human ES cell clone was hand-picked and expanded before cell enumeration assays. Cell number of lumenal cystic tissues was manually counted by blinded observers using 3D reconstructed z-stack images recorded by confocal microscopy at t=0 h, 24 h and 36 h under different microfluidic experimental conditions.
Morphogenetic quantification. Morphogenetic quantifications, including equivalent cyst diameter, embedded cyst perimeter percentage and amniotic ectoderm-like tissue thickness, were performed manually with AxioVision (Carl Zeiss MicroImaging) using confocal images recorded at the central focal plane of each cyst (40 μm above the microfluidic device bottom surface). Equivalent cyst Letter RESEARCH diameter was calculated as the average of the longest and shortest axis of each cyst. Thickness of amniotic ectoderm-like tissue was quantified as the thickness of the thinnest amniotic ectoderm-like tissue region. Embedded cyst perimeter percentage was calculated as the ratio between the perimeter of lumenal cyst embedded in Geltrex matrix and the total cyst perimeter.
Enumeration of hPGCLCs. Cells double positive for TFAP2C and SOX17 (TFAP2C⁺SOX17⁺) were identified as hPGCLCs 21,24,25. The amniotic ectoderm-like compartment was first identified as the flattened, single-cell layer tissue at t=28, 30 and 36 h or the part of embryonic-like sacs between supporting posts at t=24 h directly exposed to BMP4 stimulation. The amniotic ectoderm-like compartment was then divided into four quadrants. The two middle quadrants furthest away from the epiblast-like pole were defined as the central amniotic ectoderm-like region (CEN-AM). The two quadrants at the junction of epiblast-like and amniotic ectoderm-like compartments were defined as the epiblast-amniotic ectoderm region (EPI-AM). For consistency, only confocal images recorded at the central focal plane of each cyst (40 μm above the microfluidic device bottom surface) were used for enumeration of hPGCLCs. Confocal images were analyzed manually by blinded observers using ImageJ to determine the numbers of single (TFAP2C⁺ or SOX17⁺), double (TFAP2C⁺SOX17+) and triple (TFAP2C⁺NANOG⁺SOX17⁺) positive cells in different compartments of embryonic-like sacs.
Transwell assays. Transwell assays were conducted using 12-min Transwells with porous polyester membrane inserts (0.4 μm pore size; Coming). The Transwell membrane insert was first incubated with 1% Geltrex diluted in DMEM/F12 (Thermo Fisher Scientific) for 1 h, hPSCs suspended in mTeSR containing 10 μM Y27632 were then seeded onto the membrane insert at a density of 30×10³ cells per cm². Eighteen hours after cell seeding, culture medium was switched to basal medium supplemented with or without BMP4 (50 ng ml⁻¹), and cells were cultured for another 48 h. At this point, culture medium was replaced with fresh basal medium before small clusters of undifferentiated hPSCs suspended in basal medium were plated onto the transwell membrane insert or the lower dish. Cells were cultured for another 48 h in basal medium with or without IWP2 (5 μM, dissolved in DMSO) before analysis.
Quantification of T. The epiblast-like compartment of P-ELS was divided into four quadrants, on the basis of their relative distance to the amniotic ectoderm-like pole. Nuclear intensity of T was determined for individual cells in each quadrant by manually selecting a small area in the cell nucleus and measuring the average fluorescence intensity using ImageJ. Care was taken to ensure that selected nuclear areas did not overlap with other nuclei. T intensity for each cell was further normalized to DAPI intensity in the same nuclear area. An average normalized T intensity for cells in each quadrant was then calculated. T intensity of each quadrant was then normalized again to the quadrant with the highest T intensity and plotted.
Signalling reporter lines. A H9 human ES cell line expressing a C-terminal fusion of the gene with mNeonGreen was generated by CRISPR-Cas9 facilitated homology directed repair (HDR). In brief, activity of guide RNAs (gRNAs) was first screened in HEK 293T cells. An active gRNA with the targeting sequence gccttgctgcttcacatgga (SEQ ID NO:1), which showed the best activity, was selected and cloned into a second modified version of px33035. For the targeting construct, approximately 1,000 bp upstream and downstream of the CRISPR-Cas9 cleavage site and T stop codon was prepared by long PCR and cloned into pBluescript II KS+ (Stratagene), which was modified by adding OriP. This targeting construct was further modified by silent mutation of the gRNA targeting sequence, removal of the natural T stop codon and insertion of a 22 amino acid glycine-serine-alanine-rich flexible linker N-terminal to mNeonGreen, in frame with the T coding sequence. To generate TCF/Lef reporter-human ES cell lines, 6× TCF/Lef-hsp68-H2B-eGFP was first amplified from a plasmid provided by A.-K. Hadjantonakis (Addgene plasmid no. 32610). The amplified PCR product was ligated into an ePiggyBac vector with a constitutively active puromycin selection cassette 34. Transfection and puromycin selection were conducted as for construction of the CAG-H2B-eGFP cell line described above. Ten clonal lines were hand-picked and further expanded. H2B-eGFP expression was confirmed by fluorescence microscopy with treatment with CHIR99021 (8 μM; Cayman Chemical) and bFGF (20 ng ml⁻¹) in E6 medium. Two clonal lines with the highest and most homogeneous fluorescence signal were selected for live imaging.
Fluorescence intensity map. Heat maps of fluorescence micrographs were generated using the matplotlib package in Python. Image masks were first generated by thresholding images of DAPI staining and isolating nuclear areas from the background. Immunostaining images were then converted to heat maps on the basis of their fluorescence intensity.
Dextran diffusion assay. Morphogen diffusion in the microfluidic device was determined using fluorescein-labelled dextran (70 kDa, Invitrogen). In brief, 10 μM fluorescein-labelled dextran was supplemented into the induction channel from t=30 h onwards during the development of P-ELS. Fluorescence intensities within the cell loading channel and induction channel were then measured using confocal microscopy at t=36 h.
scRNA-seq and data analysis. P-ELS at t=48 h were treated with Accutase for 1 h to obtain single-cell suspensions. Cells from six microfluidic devices were collected and pooled into PBS containing 0.5% BSA. Transwell-AMLCs were obtained by treating human ES cells with BMP4 (50 ng ml⁻¹) for 48 h using the Transwell method. Transwell-AMLCs were treated with Accutase for 1 h to obtain single-cell suspensions. human ES cells maintained on standard tissue culture plates were dissociated into single cells using Accutase for 1 h. Transwell-AMLCs and human ES cells were counted before being mixed at a 2:1 ratio as a single-cell suspension. Within 1 h after cell dissociation, cells were loaded into the 10× Genomics Chromium system. 10× Genomics v.2 libraries were prepared according to the manufacturer's instructions. Libraries were then sequenced with a minimum coverage of 50,000 raw reads per cell on an Illumina HiSeq 4000 with paired-end sequencing. scRNA-seq data were aligned and quantified using Cell Ranger Single-Cell Software Suite (v.3.0.0, 10× Genomics) against the hg19 human reference genome. Merging of scRNA-seq data and cell clustering was performed using the Seurat R package (v.3.0.0.9). 36,37 Default setups were used unless noted otherwise. In brief, cells with nfeature_RNA≤3,200 or ≥6,200 (P-ELS), nfeature_RNA≤3,600 or ≥6,400 (Transwell-AMLC and human ES cell), or cells in which the total mitochondria' gene expression exceeded 6% of total gene expression were discarded from analysis. Gene expression was calculated by normalizing the raw count by the total count before being multiplied by 10,000 and log-transformed. After cell-cycle regression, principal component analysis was performed using the RunPCA function in Seurat. Identification of cell clusters by a shared nearest neighbor (SNN) modularity optimization based clustering algorithm was achieved using the FindClusters function with a resolution set at 0.3. Dimensionality reduction using t-SNE was generated using RunTSNE (dim=1:15). Differentially expressed genes (DEGs) were identified using FindAllMarkers, with a minimal fold difference of 0.25 in the logarithmic scale and ≥25% detection rate in either of two cell types under comparison. Violin plots were generated using VlnPlot in the Seurat R package. Heat maps were plotted on the basis of relative expression (Z-score) of top-20 gene signatures to distinguish each cell cluster. GO analyses were performed using DAVID Bioinformatics Resources 6.8 on the basis of DEGs. For comparison with published data, gene expression data obtained from different platforms (GEO repository, NCBI) were first transformed into log2(reads per million mapped reads (RPM)+1). Average expression level of each cell type was used for calculation of correlation coefficient and heat map plotting.

### Results

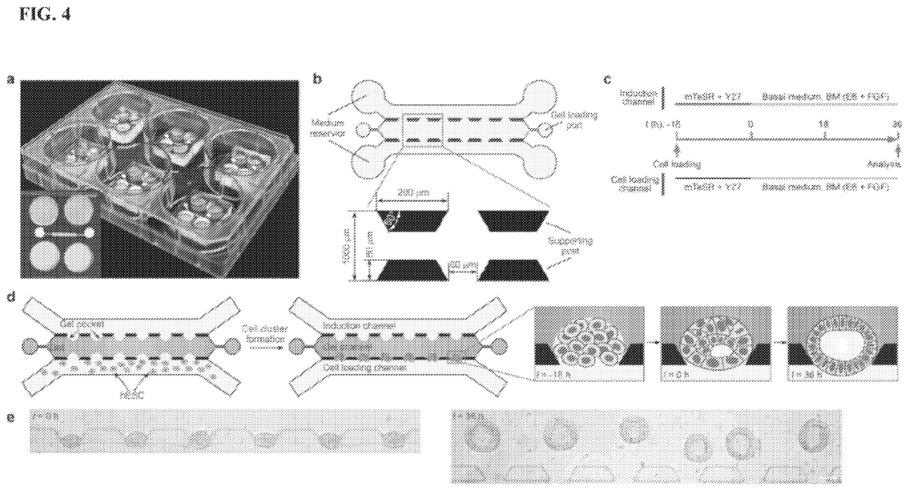

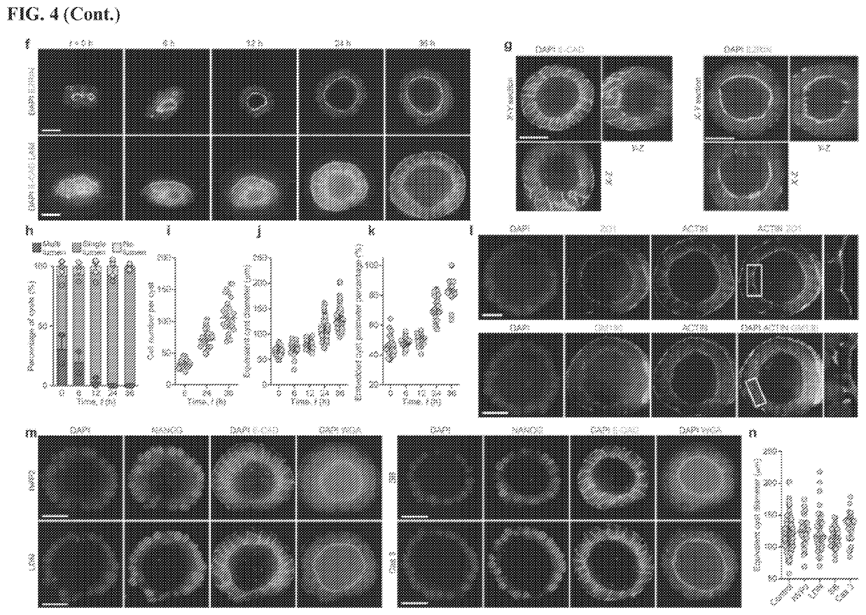

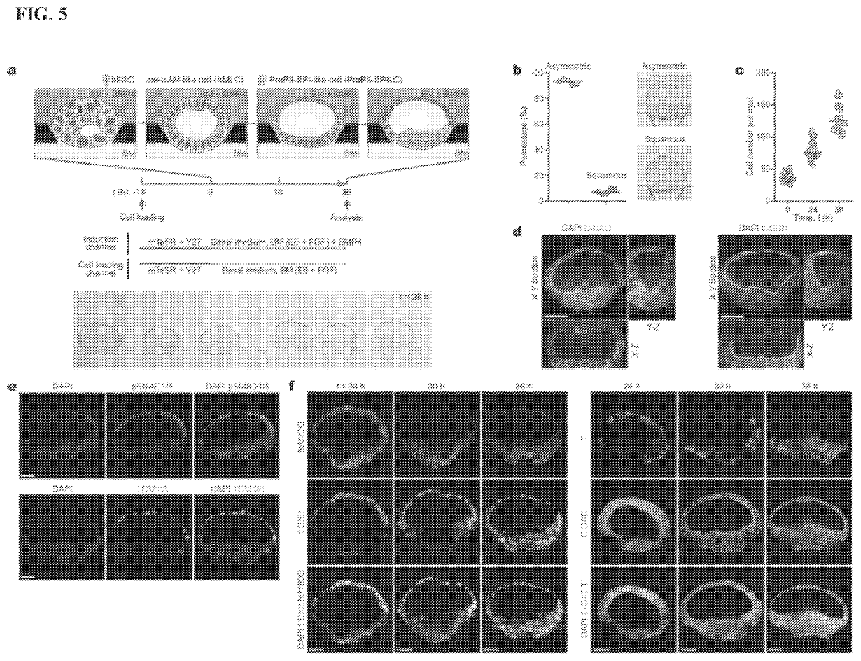

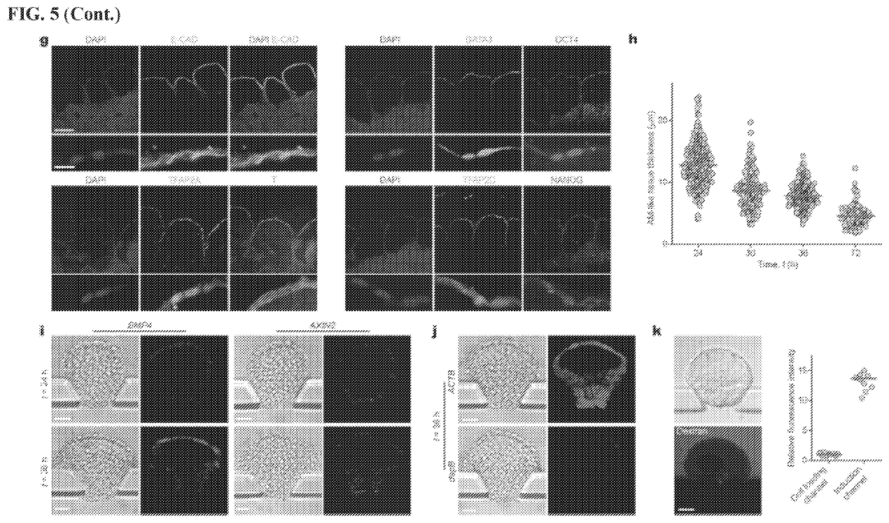

The first morphological milestone of the post-implantation human embryo is the apical-basal polarization and lumenogenesis of the epiblast, resulting in the pro-amniotic cavity 4,5,18 (FIG. 1a). Lumenogenesis of the mouse epiblast is shown to occur after the naive-to-primed pluripotency transition in the mouse epiblast 18. Thus, lumenal cysts were first generated using primed hPSCs. A microfluidic device containing three parallel channels, partitioned by evenly spaced supporting posts (FIG. 1b, FIG. 1a-c and Methods) was used. The central gel channel is preloaded with Geltrex, whereas the other two open channels serve as a cell-loading channel and a chemical-induction channel, respectively. Geltrex contraction during gelation leads to formation of concave gel pockets between supporting posts (FIG. 4d). Single H9 human embryonic stem (ES) cells injected into the cell-loading channel settle into gel pockets and subsequently cluster (FIG. 4d). At 18 h after cell seeding (designated as t=0 h), the culture medium is switched to basal medium, comprising Essential 6 medium supplemented with FGF2. From t=0 h onwards, nascent cavities containing ezrin+apical membranes emerge 5,19 (FIG. 4e, f, FIG. 1b). By t=36 h, E-cadherin⁺ epithelial sacs containing a single central lumen are developed, enclosed by a single layer of columnar, OCT4⁺NANOG⁺SOX2³⁰ epiblast-like cells (EPILCs) (FIG. 1b, c, FIG. 4e-1), reminiscent of the pro-amniotic cavity formed in the epiblast at Carnegie stage 5a 4,5,18. During lumenogenesis, epiblast-like cysts expand in size while increasing cell number (FIG. 4f, i-k). Development of epiblast-like cysts is not sensitive to inhibition of Wnt, BMP or TGF-β signalling or apoptosis (FIG. 4m, n).

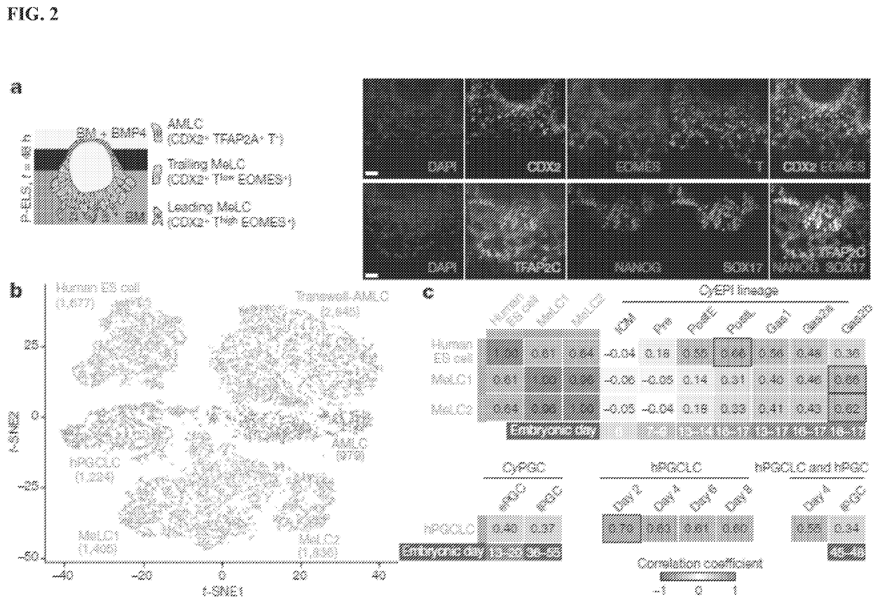

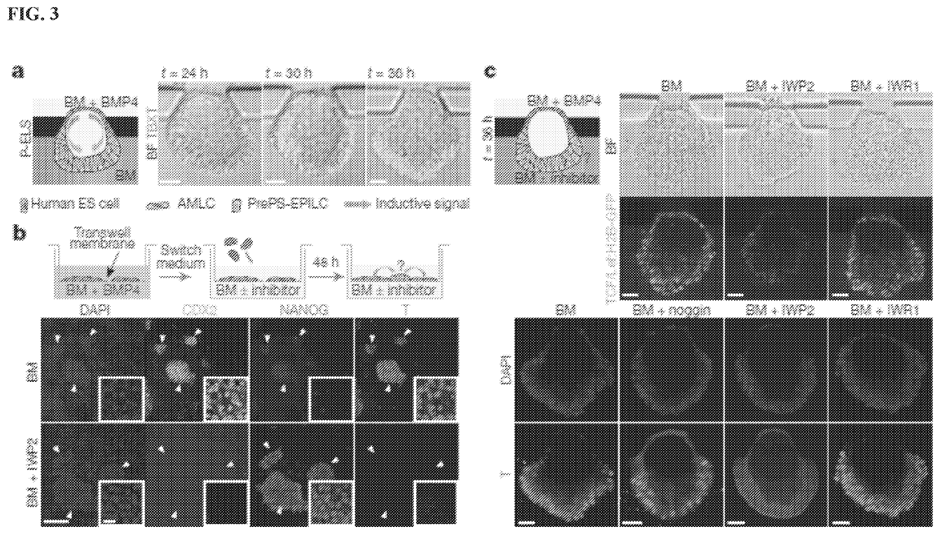

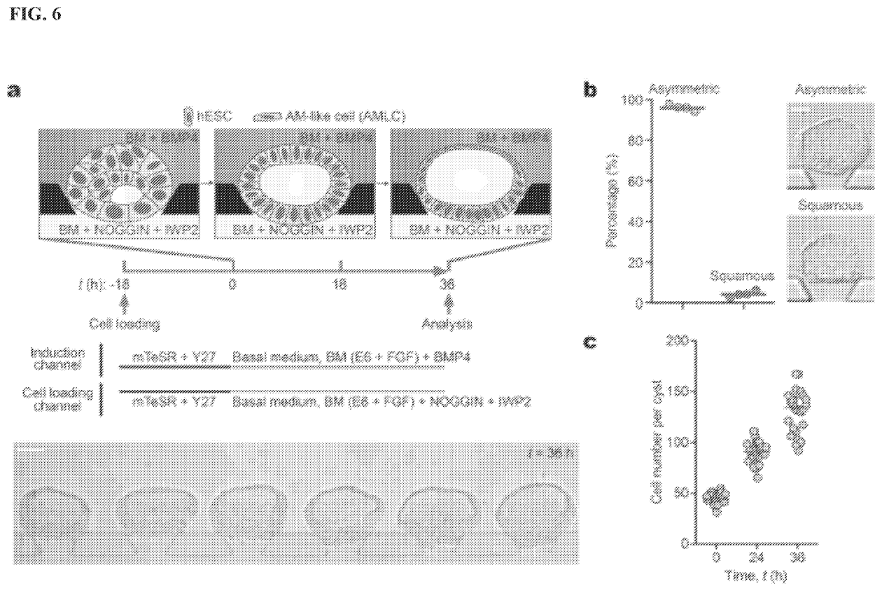

In post-implantation human embryo, formation of the pro-amniotic cavity in the epiblast is followed by dorsal-ventral patterning, resolving the epiblast into a bipolar embryonic sac, with squamous amniotic ectoderm and epiblast at the prospective dorsal and ventral poles, respectively (FIG. 1a). Formation of the bipolar embryonic sac precedes the gastrulation of the epiblast, during which the primitive streak emerges at the prospective posterior end of the epiblast (PrePSEPI) (FIG. 1a). It was tested whether epiblast-like cysts could recapitulate events similar to dorsal-ventral patterning of the epiblast. BMP4 was supplemented in the induction channel from t=0 h onwards (FIG. 1b and FIG. 5a). At t=36 h, 92% of cysts differentiated into asymmetrical, E-cadherin⁺ sacs, with a single layer of flattened, amniotic ectoderm-like cells (AMLCs) at the pole exposed to BMP4, and a stratified, epiblast-like epithelium at the other pole, resembling the human asymmetric embryonic sac before the onset of gastrulation at 7-12 days post-fertilization (Carnegie stage 5b-5c) (FIG. 1d, FIG. 5b-d). Immunofluorescence analyses confirmed nuclear staining of phosphorylated SMAD1-SMAD5 (pSMAD1/5), a downstream target of BMP signalling, in AMLCs (FIG. 5e). TFAP2A, a putative amniotic ectoderm marker 12, is exclusively expressed in AMLCs (FIG. 1d, FIG. 3e). CDX2 is thought to be a marker for both amniotic ectoderm 12 and posterior primitive streak 22, whereas brachyury (also known as T-box transcription factor or T) is a primitive streak marker 23 and is expressed transiently in the M. fascicularis amniotic ectoderm 21 and during amniogenic differentiation of hPSCs12,15. At t=24 h, incipient AMLCs express NANOG, CDX2 and T (FIG. 5f), reflecting a fate transition from pluripotent epiblast to amniotic ectoderm. Thereafter, whereas incipient AMLCs acquire squamous morphology and lose NANOG and T expression, CDX2 and T expression spreads into the epiblast-like compartment (FIG. 5f). At t=36 h, T is exclusively expressed in EPILCs, whereas NANOG is only retained at the centre of the T⁺, epiblast-like pole (FIG. 1d, FIG. 5f). Since EPILCs are CDX2⁺T⁺, but are losing NANOG supporting a PrePS-EPI phenotype exiting from pluripotency 21,22—these asymmetric sacs are hereafter referred to as posteriorized embryonic-like sacs (P-ELS). From t=36 h onwards, EPILCs start to emigrate only from the epiblast-like, pole, leading to cyst collapse. Prolonged culture to t=72 h was occasionally successful; in such cases, continuous thinning of AMLCs was evident (FIG. 2g, h), and GATA3, another putative amniotic ectoderm maker 12, appeared exclusively in TFAP2A⁺AMLCs (FIG. 5g). OCT4 showed strong nuclear staining in all cells of P-ELS (FIG. 1d, FIG. 5g), and in situ hybridization of BMP4 and AXIN2 mRNA revealed very weak expression of AXIN2 in P-ELS and robust BMP4 expression in AMLCs (FIG. 5i-k). Prominent BMP4 expression is also evident in M. fascicularis amniotic ectoderm at embryonic day (E)11-E12 21.

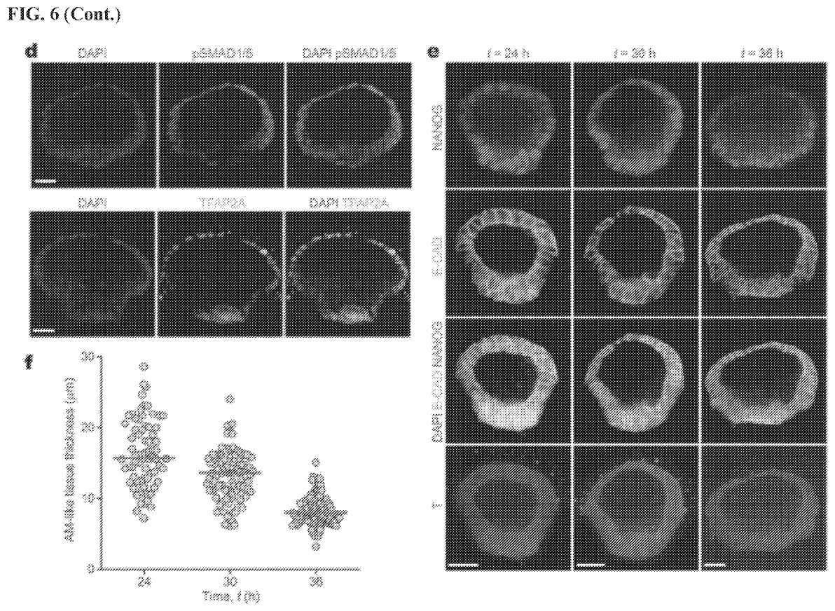

In the E11-E12 M. fascicularis embryo, T⁺ gastrulating cells emerge in the PrePS-EPI, whereas the anterior epiblast remains OCT4⁺NANOG⁺, but T⁻ 21. In the E6.5 mouse embryo, Wirt and NODAL antagonists secreted by the anterior visceral endoderm restrict the primitive streak formation to the PrePS-EPI2. To prevent gastrulating cell development, IWP2 (an inhibitor of Wnt-ligand secretion) and noggin (a BMP antagonist) were added to the cell-loading channel in addition to BMP4 in the induction channel (FIG. 1b, FIG. 6a). BMP4 stimulation still elicits patterning in 96% of cysts at t=36 h to confer amniotic ectoderm-like fate on cells directly exposed to BMP4 (FIG. 1e, FIG. 6b-d). However, compared with P-ELS, the epiblast-like pole appears more organized and is OCT4⁺NANOG⁺, but T⁻ (FIG. 1e, FIG. 6e, f). These events reflect aspects of the formation of anteriorized embryonic sac; these structures are thus designated anteriorized embryonic-like sacs (A-ELS).

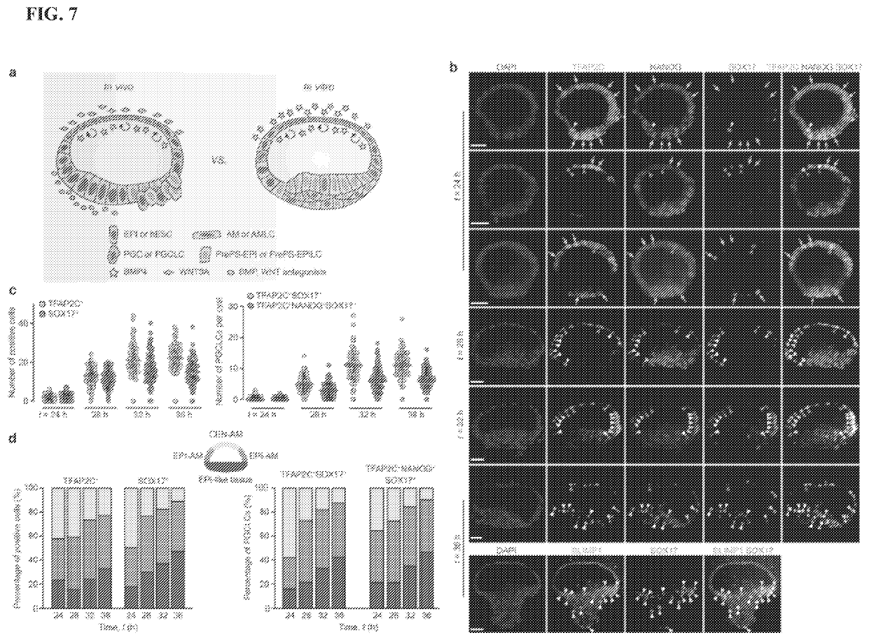

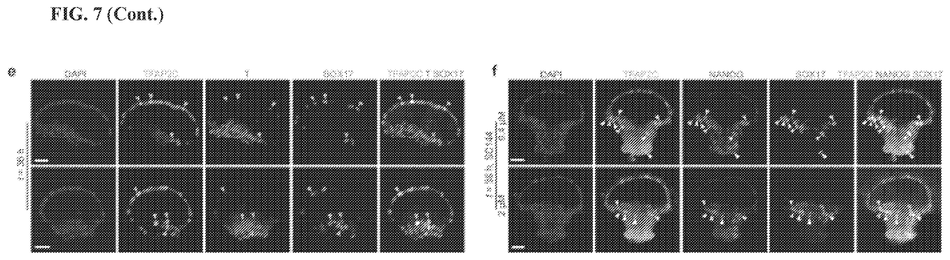

In the M. fascicularis embryo, primordial germ cells (PGCs), the precursors of sperm and egg, are thought to arise during embryonic sac formation before gastrulation 21,24 (FIG. 1a, FIG. 7a). It was examined whether human PGC-like cells (hPGCLCs) would develop in P-ELS. TFAP2C⁺SOX17⁺ cells have been identified as early-stage hPGCLCs 21,25,26. Indeed, TFAP2C⁺SOX17⁺ and SOX17⁺ TFAP2C⁻ cells, presumably in the process of hPGCLC specification, as well as TFAP2C⁺SOX17⁺ and TFAP2C⁺NANOG⁺SOX17⁺ hPGCLCs were detected at t=24 h in a scattered fashion throughout the entire P-ELS (FIG. 1d, FIG. 7b-d). At t=24 h, both TFAP2C⁺ SOX17⁺ and TFAP2C⁻NANOG⁺SOX17⁺ hPGCLCs were predominantly located in the incipient amniotic ectoderm-like compartment (FIG. 7b, d). Over time, hPGCLCs accumulated at the junction of amniotic ectoderm-like and epiblast-like compartments and in the epiblast-like pole. At t=36 h, hPGCLCs, which were prevalent in the PrePS-EPI-like pole, were also BLIMP1⁺, supporting a fully committed stage to germline cell development (FIG. 1d, FIG. 7b, d). At t=72 h, no TFAP2C⁺ or NANOG⁺ cells were detected in the amniotic ectoderm-like compartment (FIG. 5g). T expression was further examined, as T is essential for mouse PGC specification 27 and is associated with hPGCLC specification 25,26. T was detected only at t=36 h in a subset of TFAP2C⁺SOX17⁺ hPGCLCs in P-ELS (FIG. 7e). Treatment of P-ELS with the GP130 inhibitor SC144 did not affect hPGCLC specification (FIG. 7f). No TFAP2C⁺SOX17⁺ hPGCLCs were detected in A-ELS.

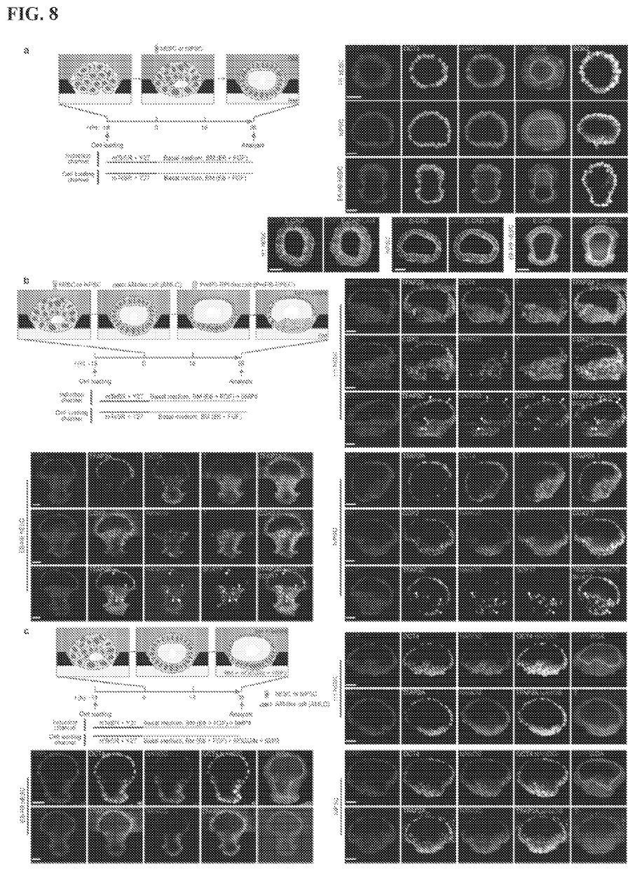

Epiblast-like cysts, P-ELS and A-ELS were generated from different primed human ES cell lines and a human induced-PSC line (FIG. 8). Specification of hPGCLCs in P-ELS was confirmed using these hPSC lines (FIG. 8b).

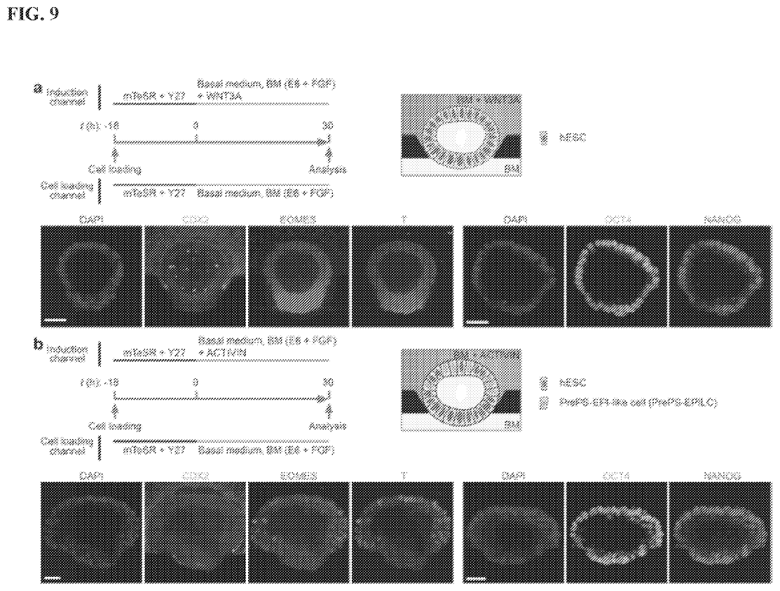

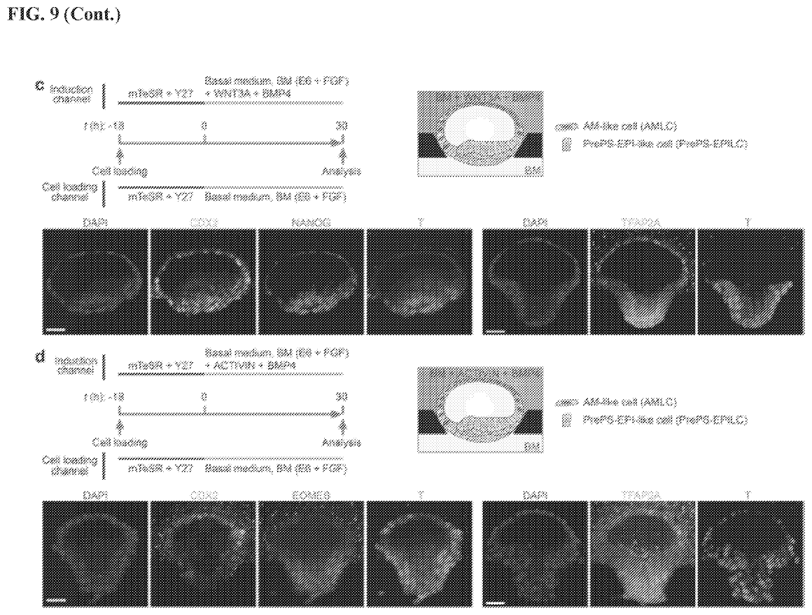

BMP and TGF-β-activin signalling have been suggested to confer characteristics of posterior 28 and anterior 29 primitive streak cell phenotypes on hPSCs, respectively; however, in P-ELS, EPILCs directly exposed to BMP4 consistently give rise to AMLCs before EPILCs at the opposite pole display a posterior primitive streak-cell phenotype (FIG. 1d). To elucidate the roles of developmental signals, WNT3A or activin A was supplemented into the induction channel, with or without BMP4 (FIG. 9). WNT3A alone gave rise to columnar epiblast-like cysts containing OCT4⁺NANOG⁺, COX2⁻EOMES⁻T⁻ EPILCs (FIG. 9a). Activin A alone generated cysts containing OCT4⁺NANOG⁺EPILCs intermixed with EOMES⁺T⁺CDX2⁻ anterior primitive streak-like cells (FIG. 9b). WNT3A and BMP4 together resulted in P-ELS (FIG. 9c), whereas activin A and BMP4 together generated asymmetric sacs containing TFAP2A⁺CDX2⁺ AMLCs at the pole directly facing activin A-BMP4 stimulation and EOMES⁺T⁺CDX2⁻ anterior primitive streak-like cells at the opposite pole (FIG. 9d). Together, these data indicate that WNT3A is dispensable for embryonic-like sac formation and confirm the ability of activin signalling to confer an anterior primitive streak-like cell phenotype on the PrePSEPI-like cells (PrePS-EPILCs).

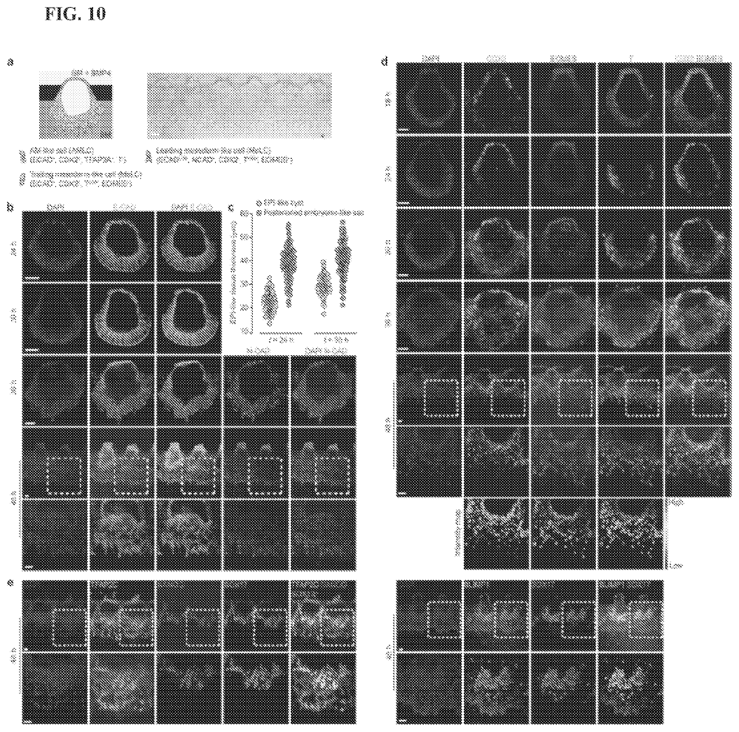

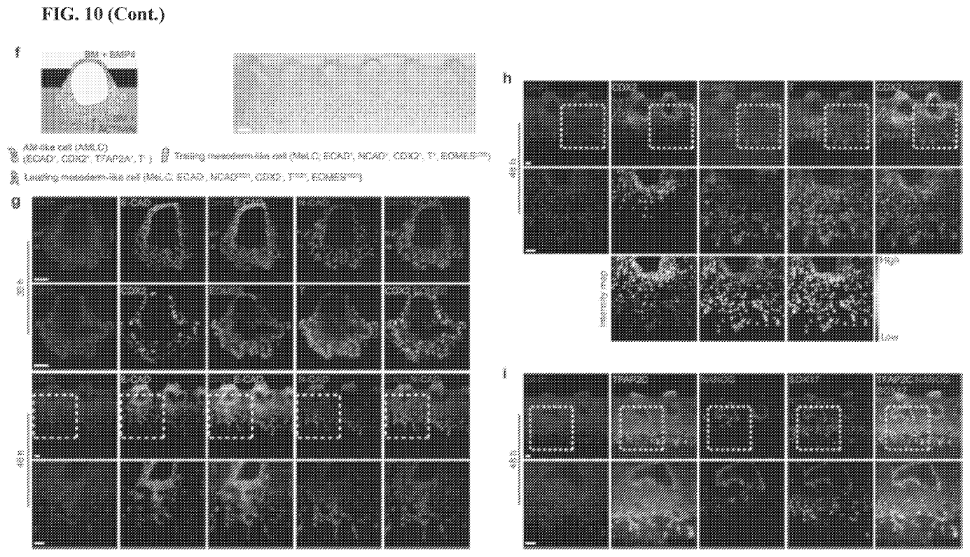

Because cell dissemination in P-ELS resembles cell movement during gastrulation of the epiblast, this event was investigated further. To this end, BMP4 was supplemented into the cell loading channel from t=0 h onwards (FIG. 2a, FIG. 10a). The PrePSEPI-like pole became thickened before cell dissemination (FIG. 10a-c). From t=36 h onwards, individual cells began to migrate away from the PrePS-EPI-like pole and morphologically acquired a mesenchymal phenotype (FIG. 10b). At t=48 h, variable levels of T, EOMES and CDX2 were detected in migrating cells; leading cells showed high intensity T staining (T^{high}) and upregulated EOMES (EOMES⁺) and N-cadherin (N-cadherin⁺), but not CDX2, whereas trailing cells exhibited CDX2 staining (CDX2⁺) and low intensity T staining (T^{low}), but did not express EOMES or N-cadherin (FIG. 2a, FIG. 10d). At t=48 h, NANOG expression was restricted to TFAP2C⁺SOX17⁺hPGCLCs, which began to migrate away from the PrePS-EPI-like compartment as clusters (FIG. 2a, FIG. 10e). To induce the anterior primitive streak-cell phenotype, activin A was supplemented into the induction channel (FIG. 10f). At t=48 h, compared with posterior primitive streak-cell phenotype, anterior primitive streak-like cells appeared more migratory, with leading cells EOMES^{high} and N-cadherin^{high} (FIG. 10g, h). NANOG expression was restricted to TFAP2C⁺SOX17⁺hPGCLCs, which were still contained in embryonic-like sacs (FIG. 10i). A distinct SOX17⁺TFAP2C⁻NANOG⁻ cell population was evident in trailing cells, which were presumably in the process of endoderm specification 30 (FIG. 10i).

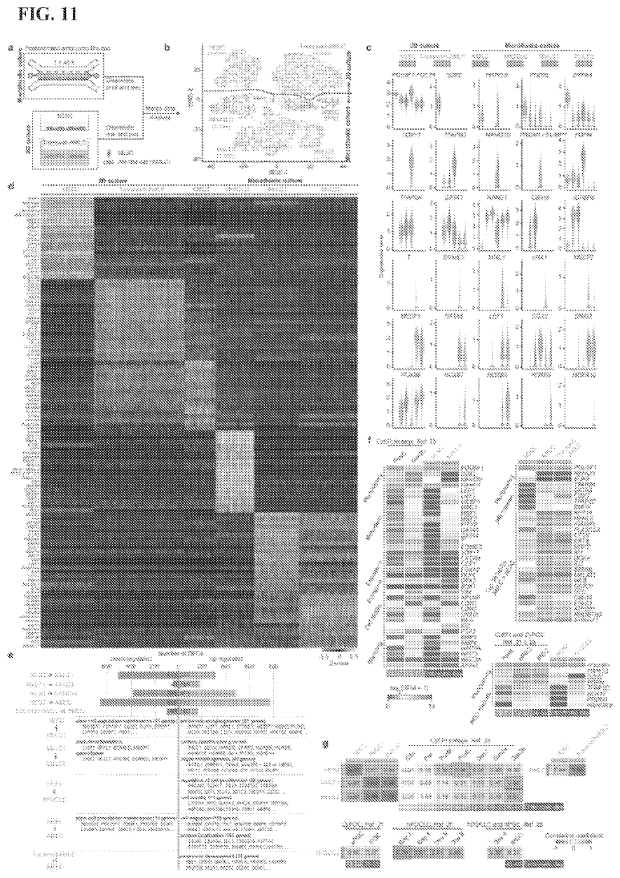

Using single-cell RNA sequencing analysis (scRNA-seq), P-ELS obtained at t=48 h, together with human ES cells and AMLCs derived using a Transwell method (Transwell-AMLC were analyzed; FIG. 11a). In the t-distributed stochastic neighbor embedding (t-SNE) plot, six distinct cell clusters emerged and were annotated as human ES cell, Transwell-AMLC, AMLC, hPGCLC, mesoderm-like cell (MeLC) 1 and MeLC2, on the basis of lineage markers (FIG. 2b, FIG. 11b). Notably, in addition to SOX17, TFAP2C, BLIMP1 (also known as PRDM1) and NANOG, the hPGCLC cluster expressed NANOS3 (FIG. 11b-f). MeLC1 and MeLC2 clusters corresponded to T^{high}EOMES⁺ leading cells and T^{low}EOMES⁻ trailing cells in P-ELC, respectively (FIG. 11b-d). Genes upregulated in the MeLC2 cluster relative to the MeLC1 cluster were enriched for those associated with ‘body pattern specification/organ morphogenesis’ (FIG. 11c-e), including HOXA10, HOXB6, HOXB8 and HOXB9, supporting a later developmental stage. Genes upregulated in the MeLC1, hPGCLC and AMLC clusters relative to the human ES cell cluster exhibit marked enrichments for ‘embryonic morphogenesis’, ‘regulation of cell proliferation/cell motility’ and ‘cell migration/protein localization’, respectively, whereas the downregulated genes are enriched for ‘stem cell population maintenance’ (FIG. 11e). Expression of ectoderm markers PAX6, SOX1 and OTX2 or endoderm markers SOX17, CXCR4, CER1 and FOXA2 was not detected in the MeLC1 cluster (FIG. 11f). Transcriptomic profiles of AMLC and Transwell-AMLC clusters were similar, and could only be distinguished using the t-SNE2 axis (FIG. 11b-g). On the basis of ontogenic M. fascicularis epiblast genes 22, human ES cell and MeLC1-MeLC2 clusters showed the closest correlations with post-implantation late M. fascicularis epiblast and gastrulating cells at E16-E17, respectively (FIG. 2c, FIG. 11g). The hPGCLC cluster exhibited the closest correlation with early-stage hPGCLCs on the basis of ontogenic M. fascicularis PGC genes21 (FIG. 2c, FIG. 11_{g}).

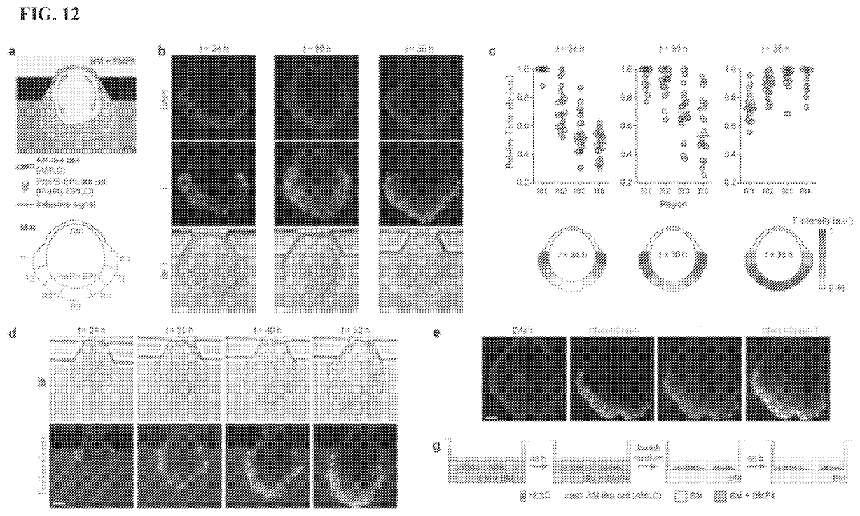

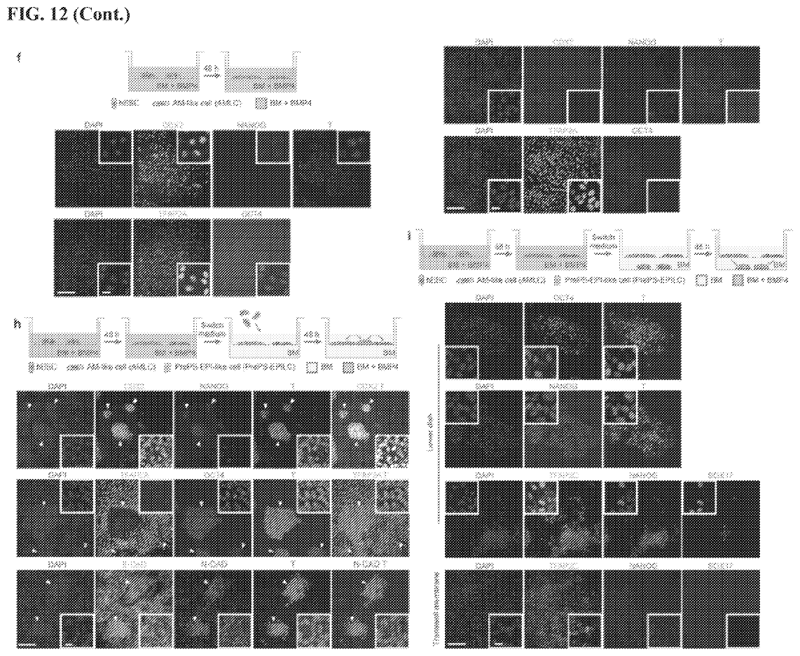

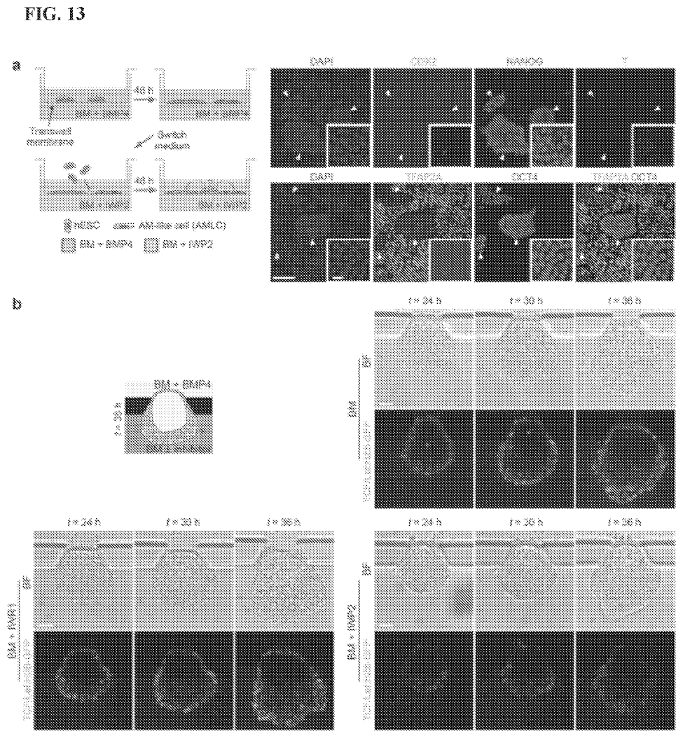

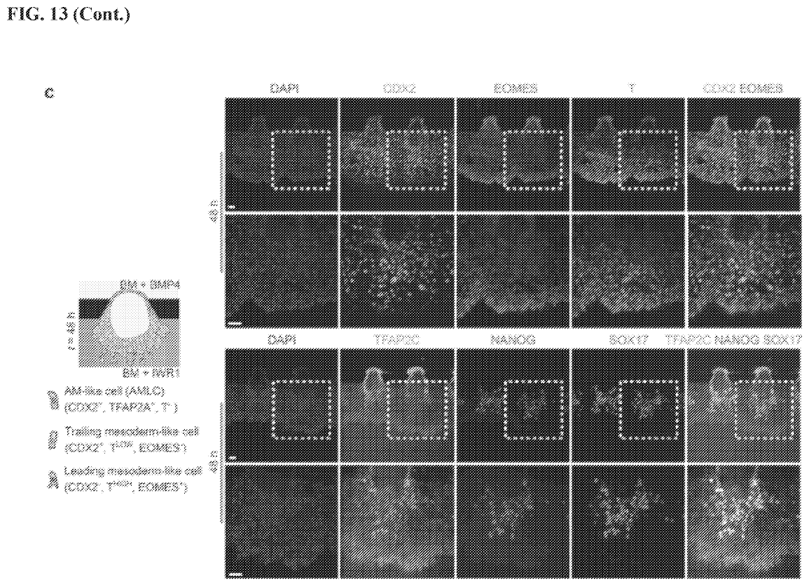

Mouse gastrulation is initiated at the proximal, posterior end of the mouse epiblast by a convergence of BMP-Wnt-NODAL signalling, established through reciprocal interactions between the mouse epiblast and the juxtaposed extraembryonic ectoderm. The trophectoderm, the counterpart of extra embryonic ectoderm in the post-implantation human embryo, however, is physically separated from the epiblast by the amniotic ectoderm. The role of AMLCs in triggering gastrulation-like events in P-ELS was investigated. T expression always emerged first in regions of the PrePS-EPI-like compartment adjacent to the amniotic ectoderm-like compartment before its propagation to the rest of the PrePS-EPI-like compartment (FIG. 3a, FIG. 12a-e). Inductive roles of Transwell-AMLCs were further examined (FIG. 3b, FIG. 12f-i). Even without exogenous inductive factors, human ES cells co-cultured with Transwell-AMLCs for 48 h would exit from pluripotency and display a posterior primitive streak-cell phenotype (FIG. 3b, FIG. 12h, i). TFAP2C⁺NANOG⁺SOX17⁺ hPGCLCs are evident in human ES cells, but not in Transwell-AMLCs, after 48 h of co-culture (FIG. 12i). Differentiation of posterior primitive streak-like cells is inhibited, however, when IWP2 is supplemented into the Transwell co-culture system (FIG. 3b, FIG. 13a), supporting the involvement of Wnt signalling in inductive effects of Transwell-AMLCs. A TCF/Lef:H2B-GFP human ES cell reporter line was used to generate P-ELS, confirming active Wnt-β-catenin signalling in the PrePS-EPI-like compartment (FIG. 3c, FIG. 13b). Of note, PrePS-EPI-like cell development is completely inhibited by IWP2 supplemented in the induction channel, but not by noggin or IWR1, a specific inhibitor targeting turnover of AXIN2, a member of the β-catenin destruction complex, indicating that initiation of gastrulation-like events in PrePS-EPI-like cells may be independent of AXIN2 (FIG. 3c, FIG. 13b). Supporting this, the ISH data (FIG. 5i) and scRNA-seq data (FIG. 11f) show very weak expression of AXIN2 in P-ELS. Prolonged culture of P-ELS to t=48 h under IWR1 treatment confirms that IWR1 does not affect the progressive development of P-ELS (FIG. 13c).

This example describes a hPSC-based microfluidic model to recapitulate successive key early human post-implantation developmental landmarks. Experiments demonstrated that AMLCs have an important role both in triggering the onset of gastrulation-like events and in the specification of hPGCLCs. The data also provide a new perspective on the fluidity of pluripotency phases associated with hPSCs and human epiblast.

### REFERENCES

1. O'Rahilly, R. & Müller, F. Developmental Stages in Embryos. (Carnegie Institution of Washington, 1987).

2. Rossant, J. Mouse and human blastocyst-derived stem cells: vive les differences. Development 142, 9-12 (2015).

3. Davidson, K. C., Mason, E. A. & Pera, M. F. The pluripotent state in mouse and human. Development 142, 3090-3099 (2015).

4. Deglincerti, A. et al. Self-organization of the in vitro attached human embryo. Nature 533, 251-254 (2016).

5. Shahbazi, M. N. et al. Self-organization of the human embryo in the absence of maternal tissues. Nat. Cell Biol. 18, 700-708 (2016).

6. Daley, G. Q. et al. Setting global standards for stem cell research and clinical translation: the 2016 ISSCR guidelines. Stem Cell Reports 6, 787-797 (2016).

7. Hyun, I., Wilkerson, A. & Johnston, J. Embryology policy: revisit the 14-day rule. Nature 533, 169-171 (2016).

8. ten Berge, D. et al. Wnt signaling mediates self-organization and axis formation in embryoid bodies. Cell Stem Cell 3, 508-518 (2008).

9. van den Brink, S. C. et al. Symmetry breaking, germ layer specification and axial organisation in aggregates of mouse embryonic stem cells. Development 141, 4231-4242 (2014).

10. Warmflash, A., Sorre, B., Etoc, F., Siggia, E. D. & Brivanlou, A. H. A method to recapitulate early embryonic spatial patterning in human embryonic stem cells. Nat. Methods 11, 847-854 (2014).

11. Harrison, S. E., Sozen, B., Christodoulou, N., Kyprianou, C. & Zernicka-Goetz, M. Assembly of embryonic and extra-embryonic stem cells to mimic embryogenesis in vitro. Science 356, eaa11810 (2017).

12. Shao, Y. et al. Self-organized amniogenesis by human pluripotent stem cells in a biomimetic implantation-like niche. Nat. Mater. 16, 419-425 (2017).

13. Xue, X. et al. Mechanics-guided embryonic patterning of neuroectoderm tissue from human pluripotent stem cells. Nat. Mater. 17, 633-641 (2018).

14. Beccari, L. et al. Multi-axial self-organization properties of mouse embryonic stem cells into gastruloids. Nature 562, 272-276 (2018).

15. Shao, Y. et al. A pluripotent stem cell-based model for post-implantation human amniotic sac development. Nat. Commun. 8, 208 (2017).

16. Rivron, N. C. et al., Blastocyst-like structures generated solely from stem cells. Nature 557, 106-111 (2018).

17. Rivron, N. et al. Debate ethics of embryo models from stem cells. Nature 564, 183-185 (2018).

18. Shahbazi, M. N. et al, Pluripotent state transitions coordinate morphogenesis in mouse and human embryos. Nature 552, 239-243 (2017).

19. Taniguchi, K. et al. Lumen formation is an intrinsic property of isolated human pluripotent stem cells. Stem Cell Reports 5, 954-962 (2015).

20. Dobreva, M. P. et al. Periostin as a biomarker of the amniotic membrane. Stem Cells Int. 2012, 987185 (2012).

21. Sasaki, K. et al. The germ cell fate of cynomolgus monkeys is specified in the nascent amnion. Dev. Cell 39. 169-185 (2016).

22. Nakamura, T. et al. A developmental coordinate of pluripotency among mice, monkeys and humans. Nature 537, 57-62 (2016).

23. Bernardo, A. S. et al. BRACHYURY and CDX2 mediate BMP-induced differentiation of human and mouse pluripotent stem cells into embryonic and extraembryonic lineages. Cell Stem Cell 9, 144-155 (2011). 24. Kobayashi, T. et al. Principles of early human development and germ cell program from conserved model systems. Nature 546, 416-420 (2017).

25. Irie, N. et al. SOX17 is a critical specifier of human primordial germ cell fate. Cell 160, 253-268 (2015).

26. Sasaki, K. et al. Robust in vitro induction of human germ cell fate from pluripotent stem cells. Cell Stem Cell 17, 178-194 (2015).

27. Aramaki, S. et al. A mesodermal factor, T, specifies mouse germ cell fate by directly activating germline determinants. Dev. Cell 27, 516-529 (2013).

28. Mendjan, S. et al. NANOG and CDX2 pattern distinct subtypes of human mesoderm during exit from pluripotency, Cell Stem Cell 15, 310-325 (2014).

29. Gadue, P., Huber, T. L., Paddison, P. J. & Keller, G. M. Wnt and TGF-β signaling are required for the induction of an in vitro model of primitive streak formation using embryonic stem cells, Proc. Natl Acad. Sci. USA 103, 16806-16811 (2006).

30. Séguin, C. A., Draper, J. S., Nagy, A. & Rossant, J. Establishment of endoderm progenitors by SOX transcription factor expression in human embryonic stem cells. Cell Stem Cell 3, 182-195 (2008).

31. Villa-Diaz, L. G. et: al. Synthetic polymer coatings for long-term growth of human embryonic stem cells. Nat. Biotechnol. 28, 581-583 (2010).

32. Watanabe, K. et al. A ROCK inhibitor permits survival of dissociated human embryonic stem cells. Nat. Biotechnol. 25, 681-686 (2007).

33. Zheng, Y. & Fu, J. Protocol for controlled modeling of human epiblast and amnion development using stem cells. Protoc. Etch. (2019). 34. Lacoste, A., Berenshteyn, F. &. Brivanlou, A. H. An efficient and reversible transposable system for gene delivery and lineage-specific differentiation in human embryonic stem cells. Cell Stem Cell 5, 332-342 (2009).

35. Cong, L. et al. Multiplex genome engineering using CRISPR/Cas systems. Science 339, 819-823 (2013). 36. Butler, A., Hoffman, P., Smibert, P., Papalexi, E. & Satija. R. Integrating single-cell transcriptomic data across different conditions, technologies, and species. Nat. Biotechnol. 36, 411-420 (2018).

37. Satija, R., Farrell, J. A., Gennert, D., Schier, A. F. &. Regev, A. Spatial reconstruction of single-cell gene expression data. Nat. Biotechnol. 33, 495-502 (2015)

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A method for preparing embryo-like tissue, comprising:
a) introducing stem cells into a microfluidic device comprising a culture channel and a plurality of fluidic channels, wherein said stem cells are introduced to said culture channel of said microfluidic device;
b) contacting said stem cells with basal medium via said plurality of fluidic channels for a defined period of time to generate said embryo-like tissue.

2. The method of claim 1, wherein said basal medium is supplemented with one or more additional components that alter BMP, WNT, YAP, and/or TGF-β signaling.

3. The method of claim 2, wherein said one or more additional components are selected from the group consisting of BMP4, noggin, and a Wnt pathway inhibitor.

4. The method of claim 2, wherein said additional components are added to said basal medium after a defined period of time.

5. The method of 2 claim 1, wherein said contacting is for 0-120 hours.

6. (canceled)

7. The method of 2 claim 1, wherein said plurality of fluidic channels comprises an induction channel and a cell loading channel.

8. The method of claim 7, wherein said induction channel and said cell loading channel comprise basal medium.

9. The method of claim 8, wherein said induction channel comprises basal medium plus BMP4 and said cell loading channel comprises basal medium.

10. The method of claim 7, wherein said induction channel comprises basal medium plus BMP4 and said cell loading channel comprises basal medium plus noggin and IWP2.

11. The method of 4 claim 1, wherein said induction channel comprises basal medium and cell loading channel comprises basal medium plus BMP4.

12. The method of 4 claim 1, wherein said induction channel comprises basal medium plus activin and cell loading channel comprises basal medium plus BMP4.

13. The method of 4 claim 1, wherein said basal medium comprises Essential 6 medium and FGF2, Essential 8 medium and FGF2, or mTesR1 medium and FGF2, or N2B27 medium and FGF2.

14. The method of 4 claim 1, wherein said culture channels comprises a plurality of posts and a gel matrix, and wherein said stem cells are located in pockets between said posts and said gel matrix.

15. The method of 4 claim 1, wherein said embryo-like tissue is a posteriorized embryonic-like sac (P-ELS) or an anteriorized embryonic-like sac (A-ELS).

16. The method of claim 15, wherein said P-ELS comprises a single layer of amniotic ectoderm-like cells at a pole of said sac exposed to BMP4 and a stratified, epiblast-like epithelium comprising PrePS-EPI-like cells at a pole exposed to basal medium.

17. The method of claim 15, wherein said A-ELS comprises a single layer of amniotic ectoderm-like cells at a pole of said sac exposed to BMP4 and a single layer of embryonic stem cells in an epiblast-like pole exposed to noggin and IWP2.

18. The method of claim 16, wherein said amniotic ectoderm-like cells express TFAP2A; said PrePS-EPI-like cells express CDX2 and T; and said epiblast-like cells express OCT4 and NANOG.

19. The method of claim 11, wherein said embryo-like tissue comprises posterior and/or anterior primitive streak-like cells.

20-26. (canceled)

27. A method of generating amniotic ectoderm-like cells, comprising:
a) introducing stem cells onto a permeable support comprising a porous membrane;
b) contacting said stem cells with basal medium plus BMP4 to generate said amniotic ectoderm-like cells.

28-29. (canceled)

30. A method of generating primitive steak-cells and/or primordial germ cell-like cells, comprising:
co-culturing amniotic ectoderm-like cells and pluripotent stem cells under conditions such that said primitive steak-cells and/or primordial germ cell-like cells are generated.

31-36. (canceled)
